(19) United States
(12) Patent Application Publication
(10) Pub. No.: US 2023/0026378 A1
(43) Pub. Date: Jan. 26, 2023

(54) ULTRA-DEFORMABLE LIPOSOMES FOR DRUG DELIVERY

(71) Applicant: Northeastern University, Boston, MA (US)

(72) Inventors: Debra Auguste, Briarcliff Manor, NY (US); Danielle Large, Boston, MA (US)

(21) Appl. No.: 17/864,077

(22) Filed: Jul. 13, 2022

Related U.S. Application Data
(60) Provisional application No. 63/221,474, filed on Jul. 13, 2021.

(51) Int. Cl. A61K 9/127; A61P 35/00; A61K 47/69

(52) U.S. Cl. CPC A61K 9/1273; A61P 35/00; A61K 47/6911

(57) ABSTRACT

Described herein are ultra-deformable liposomes comprising a first lipid and second lipid. The first lipid comprises a first hydrophilic head linked to a first aliphatic tail, and the second lipid comprises a second hydrophilic head linked to a second aliphatic tail having at least two carbons less than the first aliphatic tail. The ultra-deformable liposomes described herein are useful, for example, as drug delivery vehicles. Accordingly, also described herein are compositions comprising an ultra-deformable liposome and a cargo, such as a drug, as well as methods for delivering a drug, such as an anti-cancer therapeutic, to a tumor.

### RELATED APPLICATION

This application claims the benefit of U.S. Provisional Application No. 63/221,474, filed on Jul. 13, 2021. The entire teachings of this application are incorporated herein by reference.

### GOVERNMENT SUPPORT

This invention was made with government support under Grant No. CA174495 awarded by the National Institutes of Health. The government has certain rights in the invention.

### BACKGROUND

Breast cancer is the second leading cause of cancer related death of women in the United States. The desmoplastic response, in which cancer-associated fibroblasts are over-activated, leading to increased extracellular matrix (primarily collagen) production, is associated with the most invasive carcinomas (Barsky, S. H., Rao, C. N., Grotendorst, G. R., & Liotta, L. A. (1982). The American journal of pathology, 108(3), 276.). Further, triple negative breast cancer (TNBC) has the highest likelihood of metastasis and poor prognosis when central fibrosis is present (Takai, K., Le, A., Weaver, V. M., & Werb, Z. (2016). Oncotarget, 7(50), 82889-8290). The desmoplastic response leads to increased tumoral solid stress, compression of blood and lymphatic vessels, increased interstitial pressure, decreased perfusion and hypoxia (Stylianopoulos, T., et al. (2012). Proceedings of the National Academy of Sciences, 109(38), 15101-15108). Accordingly, the desmoplastic response presents a significant challenge to the delivery of anti-cancer therapeutics.

Chemotherapeutics, either encapsulated or free drug, are the current standard of care for triple negative breast cancer. Limitations in efficacy are primarily attributed to low drug accumulation, heterogeneous drug dispersion, and slowed drug release from liposomal carriers (Seynhaeve, A. L et al., Journal of controlled release, 172, 330-340 (2013)). Deformable particles, e.g., those with fluid membranes capable of squeezing through small pores, may be uniquely suited to penetrate and deliver drugs to desmoplastic tumors. Liposomes, which may be altered to increase the fluidity of the membrane, may represent a potential method for delivery of anti-cancer therapeutics.

Accordingly, there is a need for ultra-deformable liposomes, e.g., those with highly fluid membranes.

### SUMMARY

Described herein are ultra-deformable liposomes (UDLs) that can be used, for example, to increase cellular uptake, tumor accumulation, or cellular uptake and tumor accumulation of a cargo.

Accordingly, provided herein are liposomes comprising a first lipid and a second lipid, wherein the liposome is ultra-deformable.

Also provided herein are a plurality of ultra-deformable liposomes having a polydispersity index of about 0.05 to about 0.1.

Also provided herein are methods for increasing cellular uptake, tumor accumulation, or cellular uptake and tumor accumulation of a cargo using the ultra-deformable liposomes disclosed herein.

Ultra-deformable liposomes (UDLs), alternatively referred to as hypo-elastic liposomes (HELP), e.g., those with highly fluid membranes, were hypothesized to overcome the challenges associated with penetrating and delivering drugs to desmoplastic tumors due to their unique capability to: (1) enhance internalization via fusion with the cell membrane; (2) increase extravasation through tumor vasculature; and (3) squeeze through small pores to permeate tissues. The UDLs described herein have demonstrated increased cancer cell internalization and transendothelial cell permeability.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments.

FIG. 1A shows the incorporation of saturated, short chain lipids in a liposome bilayer.

FIG. 1B shows differences in liposomal stiffness as a function of different molar compositions of lipids in the bilayer.

FIG. 2A is a graph showing uptake of DOPC-DDPC liposomes by TNBC cells MDA-MB-231, 4T1, MCF-10A, and EpH-4Ev cells.

FIG. 2B is a graph showing uptake of DPMPC-DDPC liposomes by TNBC cells MDA-MB-231, 4T1, MCF-10A, and EpH-4Ev cells.

FIG. 2C is a graph showing uptake of various liposomes and their polyethylene glycol analogs by TNBC cells MDA-MB-231, 4T1, MCF-10A, and EpH-4Ev cells.

FIG. 3 is a graph showing transendothelial permeability of various liposome compositions.

FIG. 4 is a graph showing TNBC cell uptake of various liposomes after cells were treated with either PBS, chlorpromazine, filipin, or Dynasore.

FIG. 5 shows penetration in 4T1 spheroids of DOPC, DPMPC, DPMPC-DHPC, and DPMPC-C10 liposomes (left to right).

FIG. 6A shows a fluorescence heatmap for tumor accumulation and biodistribution of DOPC, DPMPC, DPMPC-DHPC, and DPMPC-DDPC liposomes in mice.

FIG. 6B is a graph showing average radian efficiency of DOPC, DOPC-DHPC, DOPC-DDPC, DPMPC, DPMPC-DHPC, and DPMPC-DDPC liposomes.

FIG. 6C is a graph showing average radians for DOPC, DOPC-DHPC, DOPC-DDPC, DPMPC, DPMPC-DHPC, and DPMPC-DDPC liposomes in the indicated tissues.

FIG. 7A shows a fluorescence heatmap for tumor accumulation and biodistribution of polyethylene glycol analogs of DOPC, DPMPC, DPMPC-DHPC, and DPMPC-DDPC liposomes in mice.

FIG. 7B is a graph showing average radian efficiency of polyethylene glycol analogs DOPC, DOPC-DHPC, DOPC-DDPC, DPMPC, DPMPC-DHPC, and DPMPC-DDPC liposomes.

FIG. 7C is a graph showing average radians for polyethylene glycol analogs DOPC, DOPC-DHPC, DOPC-DDPC, DPMPC, DPMPC-DHPC, and DPMPC-DDPC liposomes in the indicated tissues.

FIG. 8A is a graph showing the cellular uptake of DOPC, DOPC:DHPC(C7), DOPC:DDPC(C10), DPMPC(C16):DHPC(C7), and DPMPC(C16):DDPC(C10) liposomes in human and murine TNBC cells after 4-hour incubation at 37° C.

FIG. 8B is a graph showing diffusion of DOPC, DOPC:C7, and DOPC:C10 through collagen gels after 2- or 3 hour incubation at 37° C.

FIG. 8C is a graph showing transendothelial permeability of DOPC and DOPC: DHPC incubated in human umbilical vein endothelial cells (HUVECs)-seeded transwells after 4 hours at 37° C.

FIG. 9 is a graph showing tumor accumulation of DOPC, DPMPC, DPMPC-DHPC, DPMPC-C10, and their polyethylene glycol analogs.

FIG. 10A is a graph showing cellular uptake of DOPC and C7 liposomes in MDA-MB-231 cells.

FIG. 10B is a graph showing cellular uptake of DOPC and C10 liposomes in MDA-MB-436 cells.

FIG. 10C is a graph showing the transendothelial permeability of DOPC and DHPC liposomes.

FIG. 10D is a graph showing uptake of DOPC and C7 liposomes after treatment with PBS, filipin, or Dynasore.

FIG. 11A is a schematic of an atomic force microscopy (AFM) apparatus, in which a laser is deflected from the metal cantilever tip and measured by a photodiode.

FIG. 11B is a sample graph showing the force curves of soft versus stiff material when applying AFM.

FIG. 12A is an image of DOPC (100 mol %) liposomes using transmission electron microscopy (TEM), imaged at 20-50k magnification, scale bar represents 100 nm.

FIG. 12B is an image of DOPC-DHPC (75:25 mol:mol) liposomes using TEM, imaged at 20-50k magnification, scale bar represents 100 nm.

FIG. 12C is an image of DOPC-DDPC (75:25 mol:mol) liposomes using TEM, imaged at 20-50k magnification, scale bar represents 100 nm.

FIG. 12D is an image of DPMPC (100 mol %) liposomes using TEM, imaged at 20-50k magnification, scale bar represents 100 nm.

FIG. 12E is an image of DPMPC-DHPC (75:25 mol:mol) liposomes using TEM, imaged at 20-50k magnification, scale bar represents 100 nm.

FIG. 12F is an image of DPMPC-DDPC (75:25 mol:mol) liposomes using TEM, imaged at 20-50k magnification, scale bar represents 100 nm.

FIG. 13A is a graph showing cell viability of TNBC cells 4T1, MDA-MB-231, and MDA-MB-436 in the presence of indicated liposomes.

FIG. 13B is a graph showing cell viability of TNBC cells 4T1, MDA-MB-231, MCF10A, and EpH4-Ev in the presence of indicated liposomes.

FIG. 14A is a graph showing stress-strain curve of a UDL obtained from MPA measurements, in which the denoted linear region of the slope is used to calculate the stretching modulus.

FIG. 14B is a graph showing the stretching modulus of UDLs composed of 100% DOPC or DPMMPC or with the addition of 25% DHPC or DDPC.

FIG. 14C is a graph showing the lysis tension of UDLs composed of 100% DOPC or DPMMPC or with the addition of 25% DHPC or DDPC.

FIG. 15A is a graph showing uptake of DOPC-DHPC liposomes by TNBC cells MDA-MB-231, 4T1, MCFF10A, and EpH-4Ev cells.

FIG. 15B is a graph showing uptake of DOPC-DDPC liposomes by TNBC cells MDA-MB-231, 4T1, MCFF10A, and EpH-4Ev cells.

FIG. 15C is a graph showing uptake of DPMPC-DHPC liposomes by TNBC cells MDA-MB-231, 4T1, MCFF10A, and EpH-4Ev cells.

FIG. 15D is a graph showing uptake of DPMPC-DDPC liposomes by TNBC cells MDA-MB-231, 4T1, MCFF10A, and EpH-4Ev cells.

FIG. 16 is a graph showing the cellular binding of ultra-deformable liposomes by TNBC cells MDA-MB-231 and 4T1 cells.

FIG. 17 is a graph showing the transendothelial penetration of UDLs.

FIG. 18A is a graph showing the spheroid accumulation of UDLs and a control liposome.

FIG. 18B is a graph showing the radial spheroid penetration of UDLs and a control liposome.

FIG. 19 is a graph showing the Stern-Volmer quenching constant for UDLs at room temperature.

FIG. 20 is a graph showing the effect of bovine serum albumin pre-incubation of liposomes on cellular internalization for 4T1 TNBC cells.

FIG. 21 is a graph showing the cellular internalization by MDA-MB-231 and 4T1 cell lines of UDLs and corresponding PEG-analogs.

FIG. 22 is a graph showing the penetration of 4T1 TNBC cells by UDLs and corresponding PEG-analogs.

FIG. 23 is a graph showing the cellular uptake of UDLs and corresponding PEG-analogs which have been incubated with bovine serum albumin by 4T1 TNBC cells.

FIG. 24 is a graph showing the comparison of tumor accumulation of non-PEGylated and PEGylated liposomes.

FIG. 25 is a graph showing the comparison of biodistribution of non-PEGylated and PEGylated liposomes.

FIG. 26 is a graph showing biodistribution of various liposomes relative to DOPC-PEG.

### DETAILED DESCRIPTION

A description of example embodiments follows.

### Liposomes

This disclosure is based, in part, on the discovery that liposomes composed of a heterogeneous mixture of unsaturated long acyl chain (e.g., having at least 14 carbon atoms in the acyl chain, such as a C16 or C18 acyl chain) lipids (e.g., phospholipids) and saturated short acyl chain (e.g., having 13 or fewer carbon atoms in the acyl chain, such as a C7 or C10 acyl chain) lipids (e.g., phospholipids) are ultra-deformable, which increases tumor accumulation and decreases clearance of the liposomes by renal and lymphatic systems, even without the use of PEG.

Provided herein is a liposome comprising a first lipid and a second lipid, wherein: the first lipid comprises a first hydrophilic head linked to a first aliphatic tail; the second lipid comprises a second hydrophilic head linked to a second aliphatic tail having at least two carbons less than the first aliphatic tail; and the liposome is ultra-deformable.

As described herein, liposomes are spherical vesicles having at least one lipid bilayer. Typically, liposomes have an aqueous interior. Liposomes may be used as a drug delivery vehicle for drugs or other compounds. In a particular embodiment of the liposomes described herein, the lipid bilayer is composed of phospholipids (e.g., two or more phospholipids, such as two, three, four, or five phospholipids), although other lipids are within the scope of the instant disclosure. Examples of phospholipids include, but are not limited to, phosphatidyl choline, phosphatidyl ethanolamine, phosphatidyl serine, phosphatidyl inositol, phosphatidyl glycerol, 1,2-diheptanoyl-sn-glycero-3-phosphocholine, 1,2-didecanoyl-sn-glycero-3-phosphocholine, 1,2-dipalmitoleoyl-sn-glycero-3-phosphocholine, and 1,2-dioleoyl-sn-glycero-3-phosphocholine, 1,2-dimyristoyl-sn-glycero-3-phosphocholine, 1,2-distearoyl-sn-glycero-3-phosphocholine, 1,2-dioleoyl-sn-glycero-3-phosphoethanolamine, 1,2-dipalmitoyl-sn-glycero-3-phospho-(1′-rac-glycerol), 1,2-distearoyl-sn-glycero-3-phosphoethanolamine and 1,2-distearoyl-sn-glycero-3-phospho-(1′-rac-glycerol). Examples of other lipids contemplated by the instant disclosure include fatty acids, glycerides (e.g., triglycerides) lipidoids (e.g., as disclosed in Akinc, A. et. al., Nature Biotechnology, Vol. 26, No. 5, May 2008, pp 561-569), and sterols.

Typically, each lipid in a lipid bilayer or a liposome described herein has a hydrophilic head linked to an aliphatic tail. The hydrophobic region between the hydrophilic heads of the lipids is known as the “bilayer gap.” This region is populated by the aliphatic tails of the lipids. It will be appreciated that the hydrophilic head and aliphatic tail may be linked via any number of functional groups and/or linking moieties. Examples of functional groups that may, taken alone or in combination with a linking moiety, link a hydrophilic head to an aliphatic tail include an ester (as, for example, in the acyl group at the sn-2 position of plasmalogens designated 1-O-(1Z-alkenyl)-2-acyl-glycerophospholipids), a ketone, and/or an ether (as, for example, in the vinyl ether group at the sn-1 position of plasmalogens designated 1-O-(1Z-alkenyl)-2-acyl-glycerophospholipids). Common linking moieties include glycerol, sulfide, or disulfide. In some aspects, a linking moiety is glycero.

As used herein, the term “ultra-deformable liposome” refers to a liposome having a fluid membrane (e.g., highly fluid membrane). Fluid membranes are typically associated with a low elastic modulus (e.g., Young's modulus, such as a Young's modulus of from about 45 kPa to about 116 MPa), stretching modulus, and/or lysis tension, e.g., lower than traditional liposomes (e.g., as exemplified by control liposomes herein). In an embodiment, the ultra-deformable liposomes have a Young's Modulus of less than about 1 MPa (e.g., in the absence of a cargo), such as less than about 45 kPa. In an embodiment, the ultra-deformable liposomes have a Young's modulus of less than about 5 MPa (e.g., when loaded with a cargo). In an embodiment, the ultra-deformable liposomes have a Young's Modulus of about 0.1 MPa, about 0.2 MPa, about 0.3 MPa, about 0.4 MPa, about 0.5 MPa, about 0.6 MPa, about 0.7 MPa, about 0.8 MPa, about 0.9 MPa, or about 1.0 MPa when no cargo is present. In some embodiments, the ultra-deformable liposomes have a Young's Modulus of about 1.5 MPa, about 2.0 MPa, about 2.5 MPa, about 3.0 MPa, about 3.5 MPa, about 4.0 MPa, about 4.5 MPa, or about 5.0 MPa when loaded with a cargo. In some embodiments, the Young's Modulus is less in the bilayer gap of the lipid bilayer than at the surface(s) of the lipid bilayer. In some embodiments, the ultra-deformable liposomes have a stretching modulus of less than about 200 mN/m, e.g., less than about 175 mN/m. In some embodiments, the ultra-deformable liposomes have a lysis tension of less than 10 mN/m, e.g., less than 5 mN/m. Methods of measuring Young's modulii, stretching modulii and lysis tension are known in the art and described herein.

In an aspect, the first hydrophilic head and the second hydrophilic head are the same. In another aspect, the first and second hydrophilic heads each comprise phosphocholine, phosphoethanolamine, phosphoinosine, phosphoserine, or phosphoglycerol (e.g., phospho-1′-glycerol). In a particular aspect, the first and second hydrophilic heads each comprise phosphocholine.

In an alternative aspect, the first hydrophilic head and the second hydrophilic head are different. In another aspect, the first hydrophilic head and the second hydrophilic head independently comprise phosphocholine, phosphoethanolamine, phosphoinosine, phosphoserine, or phosphoglycerol (e.g., phospho-1′-glycerol).

In another aspect, the first hydrophilic head, the second hydrophilic head, or the first and second hydrophilic head further comprise a polyethylene glycol group. In an embodiment, the polyethylene glycol group has a molecular weight of about 350, about 750, or about 2000 g/mol. In an embodiment, the polyethylene glycol group is about 45 subunits, about 16 subunits, or about 7 subunits in length, wherein a subunit refers to —OCH₂CH₂—. In a particular embodiment, a lipid comprising a polyethylene glycol group (or a polyethylene glycol-containing lipid) is DPPE-PEG₂₀₀₀.

“Aliphatic,” as used herein, refers to an unsubstituted non-aromatic, branched or straight-chain (linear) hydrocarbon radical. In some aspects, aliphatic is branched. In some aspects, aliphatic is straight-chain (linear). “(C₁-C₁₀)aliphatic,” for example, refers to an aliphatic radical having from one to 10 carbon atoms. In some embodiments, aliphatic is (C₁-C₅₀)aliphatic, for example, (C₁₄-C₃₅)aliphatic, (C₁₄-C₂₁)aliphatic, (C₁₅-C₂₀)aliphatic, (C₁₆-C₁₈)aliphatic, (C₄-C₁₃)aliphatic, (C₆-C₁₂)aliphatic, or (C₇-C₁₀)aliphatic. “Aliphatic” can be saturated or contain one or more units of unsaturation. In some aspects, aliphatic is saturated. In some aspects, aliphatic is unsaturated (e.g., monounsaturated, polyunsaturated). Examples of aliphatic include alkyl, alkenyl and alkynyl. In some embodiments, aliphatic is alkyl, alkenyl or alkynyl (e.g., alkyl or alkenyl). In some aspects, aliphatic is alkyl. In some aspects, aliphatic is alkenyl.

As used herein, the term “alkyl” refers to a saturated aliphatic, as that term is described herein. Examples of alkyl include, but are not limited to, methyl, ethyl, n-propyl, i-propyl, n-butyl, i-butyl, s-butyl, t-butyl, n-pentyl, n-hexanyl, n-heptanyl, n-octanoyl, 1-methylbutyl, 2-methylbutyl, 3-methylbutyl, neopentyl, 3,3-dimethylpropyl, hexyl, decyl, and 2-methylpentyl.

The term “alkenyl”, refers to aliphatic, as that term is described herein, having at least one (e.g., one, two, three, four, five, etc.) carbon-carbon double bonds. Examples of alkenyl include, but are not limited to, ethenyl, vinyl, allyl, octenyl, decenyl, (E)-pentadec-7-ene, and (E)-heptadec-8-ene.

The term “alkynyl” refers to aliphatic, as that term is described herein, having at least one (e.g., one, two, three, four, five, etc.) carbon-carbon triple bond.

In an aspect, the first aliphatic tail is unsaturated. In an aspect, the first aliphatic tail is saturated. In some aspects, the first aliphatic tail has from 13 carbon atoms to 35 carbon atoms, e.g., from 13 carbon atoms to 21 carbon atoms, from 15 carbon atoms to 20 carbon atoms, from 15 carbon atoms to 18 carbon atoms, or 13, 14, 15, 16, 17, 18, 19, 20, 21, 22, 23, 24, 25, 26, 27, 28, 29, 30, 31, 32, 33, 34, or 35 carbon atoms. In some aspects, the carbon atoms in the first aliphatic tail are in a linear arrangement. In another aspect, the first aliphatic tail is unsaturated and has from 13 carbon atoms to 35 carbon atoms in a linear arrangement. In another aspect, the first aliphatic tail is unsaturated and has from 13 carbon atoms to 21 carbon atoms in a linear arrangement. In an aspect, the first aliphatic tail in unsaturated and has 13, 14, 15, 16, 17, 18, 19, 20, 21, 22, 23, 24, 25, 26, 27, 28, 29, 30, 31, 32, 33, 34, or 35 carbon atoms in a linear arrangement. In a particular aspect, the first aliphatic tail is unsaturated and has 15 or 17 carbon atoms in a linear arrangement.

In an aspect, the first lipid is 1,2-dipalmitoleoyl-sn-glycero-3-phosphocholine, 1,2-dimyristoyl-sn-glycero-3-phosphocholine, 1,2-distearoyl-sn-glycero-3-phosphocholine, 1,2-dioleoyl-sn-glycero-3-phosphoethanolamine, 1,2-dipalmitoyl-sn-glycero-3-phospho-(1′-rac-glycerol), 1,2-distearoyl-sn-glycero-3-phosphoethanolamine, 1,2-distearoyl-sn-glycero-3-phospho-(1′-rac-glycerol), or 1,2-dioleoyl-sn-glycero-3-phosphocholine. In a further aspect, the first lipid is 1,2-dipalmitoleoyl-sn-glycero-3-phosphocholine or 1,2-dioleoyl-sn-glycero-3-phosphocholine. In another aspect, the first lipid is 1,2-dimyristoyl-sn-glycero-3-phosphocholine, 1,2-distearoyl-sn-glycero-3-phosphocholine, 1,2-dioleoyl-sn-glycero-3-phosphoethanolamine, 1,2-dipalmitoyl-sn-glycero-3-phospho-(1′-rac-glycerol), 1,2-distearoyl-sn-glycero-3-phosphoethanolamine or 1,2-distearoyl-sn-glycero-3-phospho-(1′-rac-glycerol).

In an aspect, the second aliphatic tail is saturated. In some aspects, the second aliphatic tail has from 4 carbon atoms to 13 carbon atoms, e.g., from 5 carbon atoms to 12 carbon atoms, from 6 carbon atoms to 10 carbon atoms, or 4, 5, 6, 7, 8, 9, 10, 11, 12, or 13 carbon atoms. In some aspects, the carbon atoms in the second aliphatic tail are in a linear arrangement. In an aspect, the second aliphatic tail is saturated, and has from 6 carbon atoms to 12 carbon atoms in a linear arrangement. In another aspect, the second aliphatic tail is saturated, and has 6, 7, 8, 9, 10, 11, or 12 carbon atoms in a linear arrangement. In a particular aspect, the second aliphatic tail is saturated, and has 6 or 9 carbon atoms in a linear arrangement.

In an aspect, the second lipid is 1,2-diheptanoyl-sn-glycero-3-phosphocholine or 1,2-didecanoyl-sn-glycero-3-phosphocholine.

In an aspect, the second aliphatic tail has from 2 carbon atoms to 15 carbon atoms, e.g., from 4 carbon atoms to 15 carbon atoms, from 5 carbon atoms to 15 carbon atoms, from 10 carbon atoms to 15 carbon atoms, from 4 carbon atoms to 10 carbon atoms, or 2, 3, 4, 5, 6, 7, 8, 9, 10, 11, 12, 13, 14, or 15 carbon atoms, less than the first aliphatic tail. In an aspect, the second aliphatic tail has 4, 5, 6, 7, 8, 9, 10, 11, 12, 13, 14, or 15 carbon atoms less than the first aliphatic tail.

In an aspect, the liposome does not comprise a polyethylene glycol group. In an aspect, the liposome does not comprise a cholesterol group. In an aspect, the liposome does not comprise a polyethylene glycol group and/or cholesterol group.

In an aspect, the mole percent ratio of the first lipid to the second lipid in the liposome is 50 or greater to 50 or less, e.g., about 65 or greater to 35 or less, about 70 or greater or about 30 or less, about 75 or greater to about 25 or less, about 80 or greater to about 20 or less, about 90 or greater to about 25 or less, or about 95 or greater to about 5 or less. In another aspect, the mole percent ratio of the first lipid to the second lipid is about 75 to about 25. In another aspect, the mole percent ratio of the first lipid to the second lipid is about 95 to about 5. In another aspect, the mole percent ratio of the first lipid to the second lipid is about 85 to about 15. In another aspect, the mole percent ratio of the first lipid to the second lipid is about 65 to about 35.

In an aspect, the liposomes described herein have a zeta potential of about −7.5 mV to about 0 mV. In another aspect, the liposomes described herein have a zeta potential of about −6 to about −2 mV. In another aspect, the liposomes described herein have a zeta potential of about −5 mV to about 0 mV. In an aspect, the liposomes described herein have a zeta potential of about −2, about −3, about −4, about −5, about −6, or about −7 mV.

As used herein, “about” means approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower), e.g., 15 percent up or down, 10 percent up or down, 5 percent up or down, 4 percent up or down, 3 percent up or down, 2 percent up or down, or 1 percent up or down.

In an aspect, the liposomes as described herein have a stretching modulus of less than about 250 mN/m, e.g., as determined by micropipette aspiration. In an embodiment, the ultra-deformable liposomes described herein have a stretching modulus of less than about 250 mN/m. In an embodiment, the ultra-deformable liposomes described herein have a stretching modulus of less than about 500 mN/m, about 450 mN/m, about 400 mN/m, about 350 mN/m, about 300 mN/m, about 250 mN/m, about 200 mN/m, about 150 mM/m, about 100 mM/m, or about 50 mM/m, e.g., as determined by micropipette aspiration.

In an aspect, the molecular density of the bilayer gap is less than molecular density at the bilayer surface.

### Compositions

As described above, the ultra-deformable liposomes described herein have demonstrated increased cancer cell internalization and transendothelial cell permeability and, therefore, are expected to enhance cellular uptake and/or tumor accumulation of cargo embedded in and/or encapsulated by the UDLs. In some aspects, the cargo is a fluorescent dye. In some aspects, the cargo is a drug, e.g., an anti-cancer therapeutic.

Accordingly, in an aspect, provided herein are compositions comprising an ultra-deformable liposome described herein and a cargo. In an aspect, the cargo is a drug. In an aspect, the drug is selected from an anti-cancer therapeutic, a vaccine, an anti-bacterial reagent, or an anti-fungal reagent. In an aspect, the cargo is a drug. In a particular aspect, the drug is an anti-cancer therapeutic.

Also provided herein, are compositions comprising a plurality of an ultra-deformable liposome described herein having a polydispersity index (PDI) of less than 0.5, e.g., less than about 0.25, less than about 0.2, less than about 0.15, or less than about 0.1. In some aspects, the plurality of UDLs has a PDI of about 0.01 to about 0.2. In an aspect, the compositions described herein have a polydispersity index of about 0.05 to about 0.1. In an aspect, the compositions described herein have about 0.01, about 0.02, about 0.03, about 0.04, about 0.06, about 0.07, about 0.08, about 0.09, about 0.1, about 0.11, about 0.12, about 0.13, about 0.14, about 0.15, about 0.16, about 0.17, about 0.18, about 0.19, or about 0.2.

Liposomes of the present disclosure are typically used in a composition (e.g., a composition comprising a liposome of the present disclosure, a cargo and, optionally, one or more pharmaceutically acceptable carriers). A “pharmaceutically acceptable carrier” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like, and combinations thereof, as would be known to those skilled in the art (see, for example, Allen, L. V., Jr. et al., Remington: The Science and Practice of Pharmacy (2 Volumes), 22nd Edition, Pharmaceutical Press (2012).

The composition can be formulated for a particular route(s) of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred route of administration can vary depending on the particular composition chosen. In some embodiments, the composition is formulated for oral administration. In some embodiments, the composition is formulated for topical administration.

Compositions of the present disclosure can be made up in a solid form (including, without limitation, capsules, tablets, pills, granules, powders or suppositories), or in a liquid form (including, without limitation, solutions, suspensions or emulsions). The compositions can be subjected to conventional pharmaceutical operations, such as sterilization, and/or can contain conventional inert diluents, lubricating agents, or buffering agents, as well as adjuvants, such as preservatives, stabilizers, wetting agents, emulsifiers and buffers, etc. Typically, the compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) binders, e.g., magnesium aluminum silicate, starch paste, gelatin, tragacanth, methylcellulose, sodium carboxymethylcellulose and/or polyvinylpyrrolidone;
d) disintegrants, e.g., starches, agar, alginic acid or its sodium salt, or effervescent mixtures; and
e) absorbents, colorants, flavors and sweeteners.
   Tablets may be either film-coated or enteric-coated according to methods known in the art.

Suitable compositions for oral administration include the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions comprise a composition of the present disclosure in the form of an aqueous isotonic solution or suspension, and certain suppositories comprising a composition of the present disclosure are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions comprising a composition of the present disclosure for topical application, e.g., to the skin and eyes, include aqueous solutions, suspensions, ointments, creams, gels or sprayable formulations, e.g., for delivery by aerosol or the like. Such topical delivery systems will, in particular, be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein, a topical application may also pertain to an inhalation or to an intranasal application. A composition suitable for inhalation or intranasal administration may be conveniently delivered in the form of a dry powder (either alone, as a mixture, for example a dry blend with lactose, or a mixed component particle, for example, with phospholipids) from a dry powder inhaler, or an aerosol spray presentation from a pressurized container, pump, spray, atomizer or nebulizer, with or without the use of a suitable propellant.

The present disclosure further provides compositions and dosage forms that comprise one or more agents that reduce the rate by which the composition of the present disclosure as an active ingredient will decompose. Such agents, which are referred to herein as “stabilizers,” include, but are not limited to, antioxidants such as ascorbic acid, pH buffers, or salt buffers, etc.

A composition of the present disclosure is typically formulated into pharmaceutical dosage forms to provide an easily controllable dosage of the cargo and to give the patient an elegant and easily handleable product. The dosage regimen for the compositions of the present disclosure will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration; the renal and hepatic function of the patient; and the effect desired. Compositions described herein, or a pharmaceutically acceptable salt thereof, may be administered in a single daily dose, or the total daily dosage may be administered in divided doses, e.g., two, three, or four times daily.

In certain instances, it may be advantageous to administer a composition of the present disclosure in combination with one or more additional therapeutic agent(s). For example, it may be advantageous to administer a composition of the present disclosure in combination with one or more additional therapeutic agents, e.g., independently selected from an anti-cancer agent, anti-allergic agent, anti-emetic, pain reliever, immunomodulator and cytoprotective agent, to treat cancer.

The term “combination therapy” refers to the administration of two or more therapeutic agents to treat a disease, disorder or condition described herein. Such administration encompasses co-administration of the therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Such administration also encompasses use of each type of therapeutic agent in a sequential manner, either at approximately the same time or at different times. The therapeutic agents in a combination therapy can be administered via the same administration route or via different administration routes. Powders and/or liquids may be reconstituted or diluted to a desired dose prior to administration. Typically, the treatment regimen will provide beneficial effects of the drug combination in treating the diseases, conditions or disorders described herein.

### Methods

As described above, the ultra-deformable liposomes my increase cellular uptake and/or tumor accumulation. Without wishing to be bound by theory, this may be possible by enhancing internalization via fusion with the cell membrane, increasing extravasation through tumor vasculature, and/or squeezing through small pores to permeate tissues.

Accordingly, also provided herein is a method for delivering a drug to a tumor, the method comprising contacting a cell or a tumor with a therapeutically effective amount of an ultra-deformable liposome cargo composition as described herein.

The term “a therapeutically effective amount,” as used herein, refers to an amount of a therapeutic agent, such as a composition of the present disclosure, that, when administered to a subject, such as a human, is sufficient to effect treatment. The amount of a therapeutic agent that constitutes an “effective amount” will vary depending on the therapeutic agent, the condition being treated and its severity, the manner of administration, the duration of treatment, or the subject to be treated (e.g., age, weight, fitness of the subject), but can be determined routinely by one of ordinary skill in the art based on his own knowledge and this disclosure. In embodiments, an “effective amount” effects treatment as measured by a statistically significant change in one or more indications, symptoms, signs, diagnostic tests, vital signs, and the like. In other embodiments, an “effective amount” manages or prevents a condition as measured by a lack of a statistically significant change in one or more indications, symptoms, signs, diagnostic tests, vital signs, and the like.

The term “tumor” refers to an abnormal mass of tissue wherein the growth of the mass surpasses and is not coordinated with the growth of a normal tissue. A neoplasm or tumor may be “benign” or “malignant,” depending, for example, on the following characteristics: degree of cellular differentiation (including morphology and functionality), rate of growth, local invasion, and metastasis.

In an aspect, the tumor is desmoplastic. In an aspect, the desmoplastic tumor is associated with breast cancer, colorectal cancer, prostate cancer, lung cancer, pancreatic cancer, renal cancer, ovarian cancer, or brain cancer.

In an aspect, the tumor is in a subject.

Also provided herein are methods of treating a disease, disorder or condition described herein (e.g., cancer) in a subject (e.g., a subject in need thereof). The methods comprise administering to the subject a composition of the present disclosure (e.g., a liposome of the present disclosure and a cargo, typically, a drug).

As used herein, the term “subject” refers to an animal. Typically, the animal is a mammal. A subject also refers to, for example, primates (e.g., humans), cows, sheep, goats, horses, dogs, cats, rabbits, rats, mice, fish, birds and the like. In certain embodiments, the subject is a human.

As used herein, a subject (e.g., a human) is “in need of” a treatment if such subject would benefit biologically, medically or in quality of life from such treatment.

“Treat,” “treating” and “treatment,” as used herein, refer to the administration of a medication or medical care to a subject, such as a human, having a disease or condition of interest, e.g., a cancer, and includes: (i) inhibiting the disease or condition, e.g., arresting its development; (ii) relieving the disease or condition, e.g., causing regression of the disease or condition; and/or (iii) relieving the symptoms resulting from the disease or condition (e.g., pain, weight loss, cough, fatigue, weakness, etc.).

Provided herein are methods for treating a cancer in a subject (e.g., a subject in need thereof), comprising administering to the subject a therapeutically effective amount of a composition of the present disclosure.

The term “cancer” refers to a class of diseases characterized by the development of abnormal cells that proliferate uncontrollably and have the ability to infiltrate and destroy normal body tissues.

A wide variety of cancers, including solid tumor cancers, are amenable to the methods disclosed herein. In some embodiments, the cancer is a solid tumor cancer (e.g., a colorectal, breast, prostate, lung, pancreatic, renal, brain or ovarian cancer). In some embodiments, the cancer comprises a solid tumor (e.g., a colorectal, breast, prostate, lung, pancreatic, renal, brain or ovarian tumor). In some embodiments, the cancer comprises a desmoplastic solid tumor (e.g., a colorectal, breast, prostate, lung, pancreatic, renal, brain or ovarian tumor). In an embodiment, the cancer is breast cancer, e.g., triple negative breast cancer (TNBC).

Other examples of cancer treatable according to the methods described herein include Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Anal Cancer; Appendix Cancer; Astrocytomas, Childhood; Atypical Teratoid/Rhabdoid Tumor, Childhood, Central Nervous System; Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood; Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sézary Syndrome); Ductal Carcinoma In Situ (DCIS); Embryonal Tumors, Central Nervous System, Childhood; Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary; Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CIVIL); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma; Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sézary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sezary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis, Transitional Cell Cancer; Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

Metastases of the aforementioned cancers can also be treated in accordance with the methods described herein.

A composition described herein can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. In some embodiments, administration is oral. In some embodiments, administration is intravenous. In some embodiments, administration is topical. The preferred mode of administration can vary depending on the particular compound or agent. Typically, a compound of the disclosure or other therapeutic agent will be administered from about 1 to about 6 (e.g., 1, 2, 3, 4, 5 or 6) times per day, also or alternatively, as an infusion (e.g., a continuous infusion).

Specific dosage and treatment regimens for any particular patient will depend, for example, upon a variety of factors, such as the activity of the specific agent employed, the age, body weight, general health status, sex, diet, time of administration, rate of excretion, drug combination, the severity and course of the disease, condition or symptoms, the subject's disposition to the disease, condition or symptoms, and the judgment of the treating physician. Determining the dosage for a particular agent, subject and disease, disorder or condition is within the abilities of one of skill in the art.

### EXEMPLIFICATION

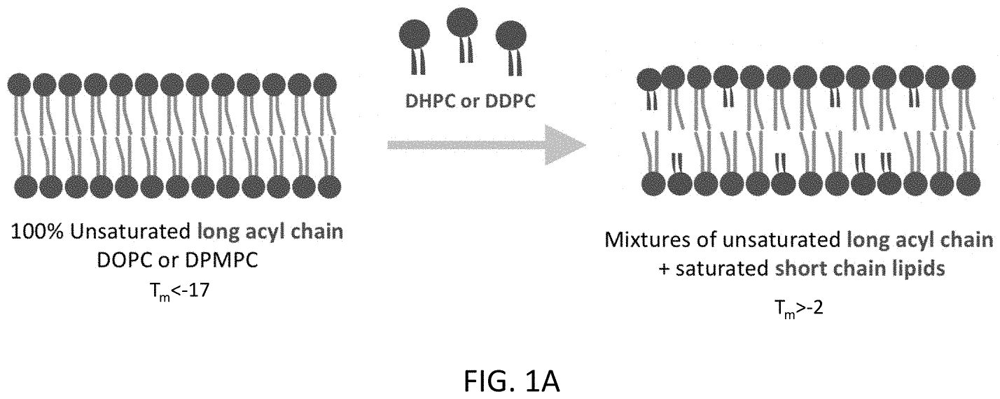

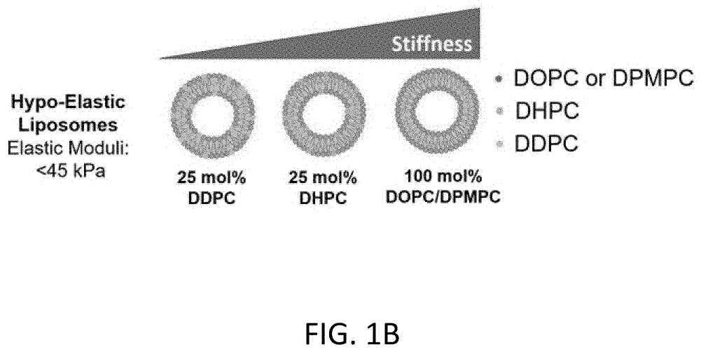

Liposomal membrane deformability and stiffness can be modulated through the incorporation of lipids with varying acyl chain length or unsaturated phospholipids (Hussain, A. et al., International journal of nanomedicine, 12, 5087 (2017)). Incorporation of phospholipids with short acyl chain length (e.g., less than 14) or multiple double bonds (e.g., polyunsaturated) may disrupt ordered packing of lipids within the bilayer, consequently increasing fluidity (FIG. 1). Herein, it has been examined how differences in membrane composition modulate cellular uptake, transendothelial permeability, and collagen diffusivity.

It has been previously demonstrated that altering nanolipogel elasticity resulted in a Young's modulus-dependent trend in cellular and tumor uptake. These data established that soft (<1.6 MPa) liposomes were internalized by cells better compared to their stiffer (>13.8 MPa) counterparts (Hussain, A. et al., International journal of nanomedicine, 12, 5087 (2017)). As stated above, this can be achieved by incorporating lipids of different acyl chain lengths or degrees of saturation, as well as the use of edge activators (e.g., surfactants).

Detailed Description of Liposomes: Liposomes were formulated with 1,2-dioleoyl-sn-glycero-3-phosphocholine (DOPC) or 1,2-dipalmitoleoyl-sn-glycero-3-phosphocholine (DPMPC) and 1,2-diheptanoyl-sn-glycero-3-phosphocholine (DHPC) or 1,2-didecanoyl-sn-glycero-3-phosphocholine (DDPC) and 1% lipophilic dye, either benzoxazolium, 3-octadecyl-2-[3-(3-octadecyl-2(3H)-benzoxazolylidene)-1-propenyl]-perchlorate (DiO) or 1,1′-dioctadecyl-3,3,3′,3′-tetramethylindotricarbocyanine iodide) (DiR) for visualization in vitro or in vivo, respectively. Liposomes were synthesized using the thin film hydration method followed by extrusion. Tables 1 and 2 describe liposome components and various characteristics of the liposomes, respectively.

TABLE 1
Liposome Components

| | | ACL: | | Acyl |
|---|---|---|---|---|
| | | Double | Tₘ | Chain |
| Lipid | Abbreviation | Bond | (° C.) | Length |
| 1,2-diheptanoyl-sn-glycero-3- | DHPC (C7) | 7:0 | <−2 | 7 |
| phosphocholine | | | | |
| 1,2-didecanoyl-sn-glycero-3- | DDPC (CIO) | 10:0 | <−2 | 10 |
| phosphocholine | | | | |
| l,2-dipalmitoleoyl-sn-glycero- | DPMPC (Cl6) | 16:1 | 41 | 16 |
| 3-phosphocholine | | | | |
| l,2-dioleoyl-sn-glycero-3- | DOPC | 18:1 | −17 | 18 |
| phosphocholine | | | | |
| l,2-dimyristoyl-sn-glycero-3- | DMPC | 14:0 | 24 | 14 |
| phosphocholine | | | | |
| 1,2-distearoyl-sn-glycero-3- | DSPC | 18:0 | 55 | 18 |
| phosphocholine | | | | |
| 1,2-dioleoyl-sn-glycero-3- | DOPE | 18:1 | −16 | 18 |
| phosphoethanolamine | | | | |
| 1,2-dipalmitoyl-sn-glycero-3- | DPPG | 16:0 | 41 | 16 |
| phospho-(l′-rac-glycerol) | | | | |
| 1,2-distearoyl-sn-glycero-3- | DSPE | 18:0 | 74 | 18 |
| phosphoethanolamine | | | | |
| 1,2-distearoyl-sn-glycero-3- | DSPG | 18:0 | 55 | 18 |
| phospho- | | | | |
| (1′-rac-glycerol) | | | | |
| lipophiliccarbocyaninedye | DiO | | | — |

*Acyl Chain Length (ACL)

TABLE 2
Liposomes

| Lipid Composition (mol %) | Size (nm) | PDI | Zeta Potential (mV) |
|---|---|---|---|
| DOPC (100) | 89.9 | 0.059 | −8.2 |
| DOPC:DHPC (75:25) | 86.9 | 0.072 | −3.4 |
| DOPC:DDPC (75:25) | 88.3 | 0.091 | −3.2 |
| DPMPC (100) | 90.1 | 0.071 | −4.3 |
| DPMPC:DHPC (75:25) | 90.8 | 0.088 | −3.4 |
| DPMPC :DDPC | 91.9 | 0.130 | −5.1 |
| DOPC:DHPC:DiO (74:25:1) | 86.9 | 0.059 | −2.9 |
| DOPC:DDPC:DiO (74:25:1) | 89.9 | 0.059 | −3.2 |
| DPMPC:DHPC:DiO (74:25:1) | 81.3 | 0.088 | −3.4 |
| DPMPC:DDPC:DiO (74:25:1) | 91.9 | 0.130 | −5.0 |
| DOPC:DiO (99:1) | 89.9 | 0.064 | −8.2 |
| DOPC:DPPE-PEG₂₀₀₀ (95:5) | 100 | 0.154 | −6.3 |
| DPMPC :DPPE-PEG₂₀₀₀ (95:5) | 103 | 0.143 | −7.5 |
| DPMPC :DDPC DPPE-PEG₂₀₀₀ | 98 | 0.165 | −7.3 |
| (70:25:5) | | | |
| DPMPC:DHPC:DPPE-PEG₂₀₀₀ | 105 | 0.172 | −6.5 |
| (70:25:5) | | | |

Example Features: The ability to permeate tissues with liposomes composed of mixtures of lipids with different acyl chain lengths and degrees of saturation is shown and described. Increased cellular uptake and tumor accumulation of the liposomes is shown and described. Increased trans-endothelial penetration of the liposomes in vitro (mimics vascular permeability) is shown and described.

Example Advantages of UDLs: Higher tumor accumulation compared to conventional liposomes. Penetration of tissues, e.g., greater penetration depth in in vitro models compared to conventional liposomes. Equivalent tumor accumulation compared to PEGylated liposomes. Lipids are relatively inexpensive. More efficient drug delivery will enable less doses of a given drug when delivered using ultra deformable liposomes.

Example Uses of UDLs: Drug delivery vehicle, e.g., for delivery of: anti-cancer therapeutics, vaccines, anti-bacterial reagents, anti-fungal reagents, or topical drug delivery (dermal application).

Atomic Force Microscopy: In the context of liposomes, atomic force microscopy (AFM) may be utilized to quantitatively characterize the elastic modulus (Young's Modulus) at the level of an individual liposome. Elastic characterization of various liposome compositions and surface modifications have been determined using AFM. Careful attention must be paid to the methods used during liposomal AFM measurements, as these measurements are only reproducible when using the same experimental set up and interpretative model. Further, special attention should be paid to the choice of cantilever tip, as sample stiffness is known to vary with differing tip geometry.

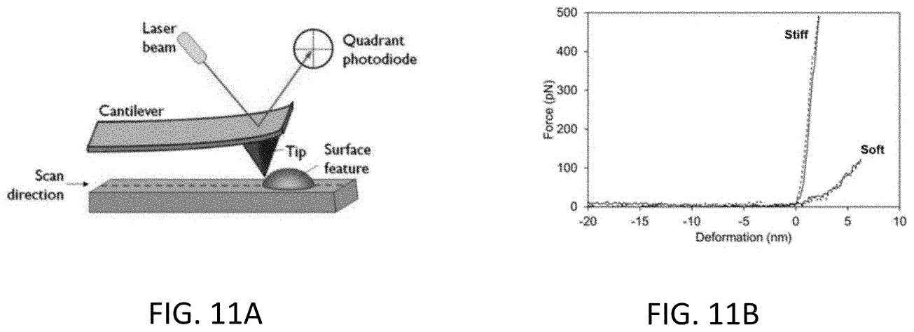

A cantilever tip (diameter 10-50 nm), typically composed of silica nitride, is used to apple a force to samples immobilized on a stiff substrate, such as mica or silicon. AFM utilizes an apparatus in which a laser beam is deflected from the tip of the cantilever and detected by a position detector (FIGS. 11A and 11B). Upon reaching a maximum force determined by the user, the cantilever retracts, and the length of deformation is reported. The Young's modulus is defined as the slope of the linear region of the stress versus strain curve generated from these measurements. These data can be fit to an elastic model, in which the Hertz equation (below) is commonly used to describe soft liposomes.

$$F = \frac{E}{( 1 - {v}^{2} )} * \frac{tan β}{\sqrt{2}} * {δ}^{2}$$

In this equation, the quasi-static force F is related to the indentation depth 6: Where 0 is the face angle, v is Poisson's ratio of the material assumed here to be 0.5 (isotropic incompressible), and E is the Young's modulus.

### Example 1

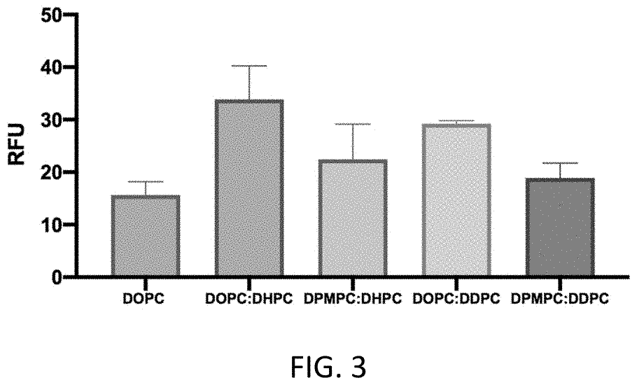

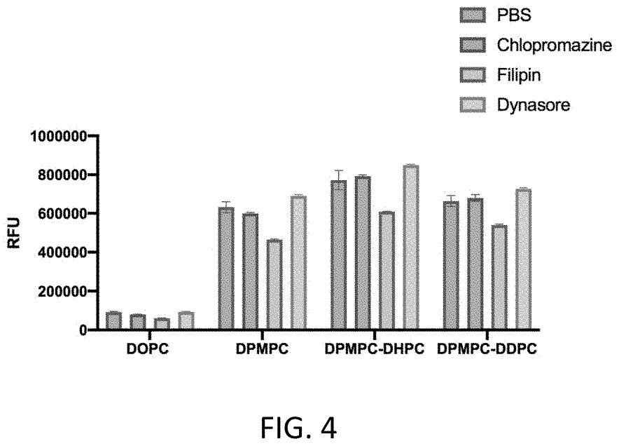

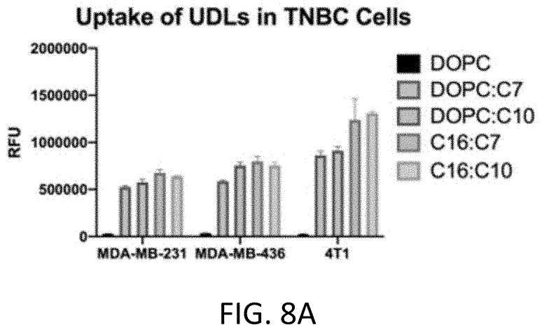

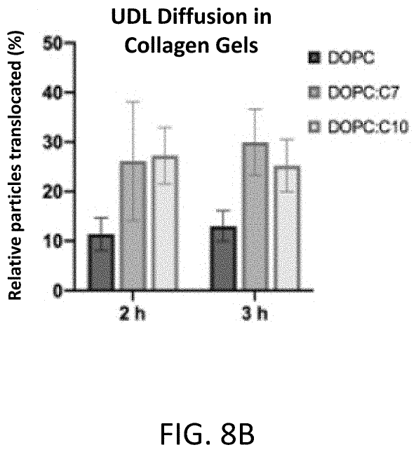

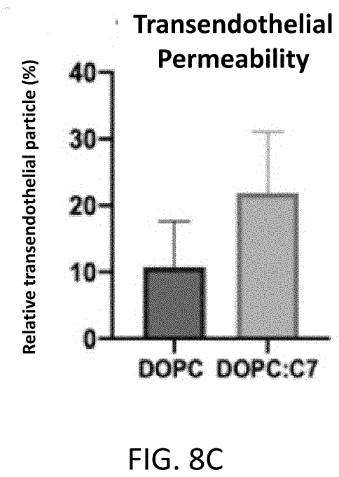

Ultra-Deformable Liposomes (UDLs) were synthesized via thin film hydration followed by extrusion using lipids listed in Table 1. Liposomes were then characterized for size, polydispersity index, and zeta potential (Table 2). For uptake studies, 5×10⁴ cells per well were cultured in a 24-well plate and incubated overnight. Cells were then treated with 200 μM lipid of liposomes and incubated for 4 hours (h) at 37° C. UDL uptake was measured by flow cytometry. Transendothelial permeability was evaluated by culturing 2×10⁴ human umbilical vein endothelial cells (HUVECs) in transwell inserts for 48 hours, followed by stimulation with TNBC conditioned media for 24 hours. UDLs (200 μM lipid) were incubated in transwells for 4 hours at 37° C. Media in the bottom of the chamber was then collected and measured for DiO fluorescence using a SpectraMax i3 fluorescence spectrophotometer (FIG. 3). UDL diffusion through collagen gels was determined by incubating UDLs in transwell inserts coated with 100 μl collagen gel (3 mg/ml) for 2-3 h (FIG. 8B).

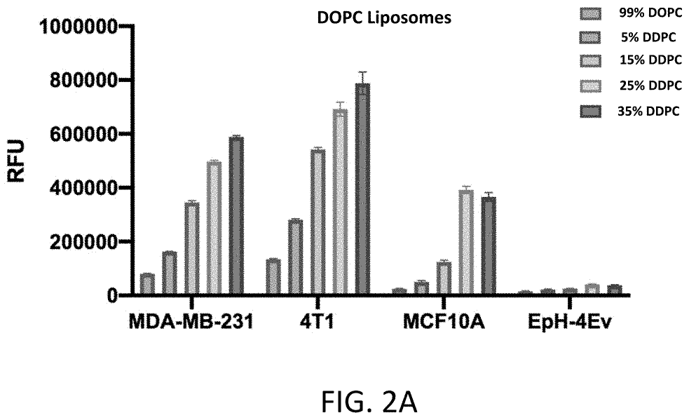

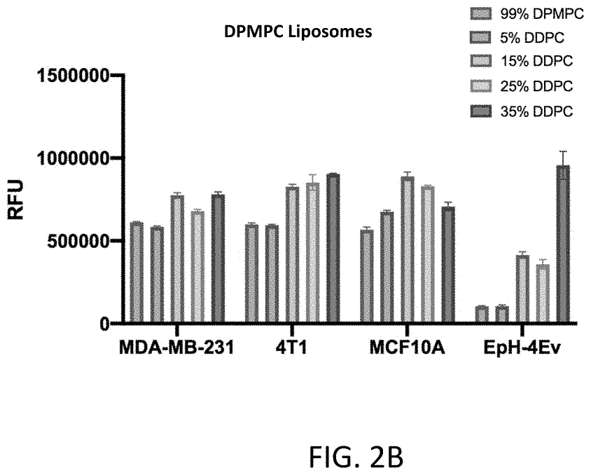

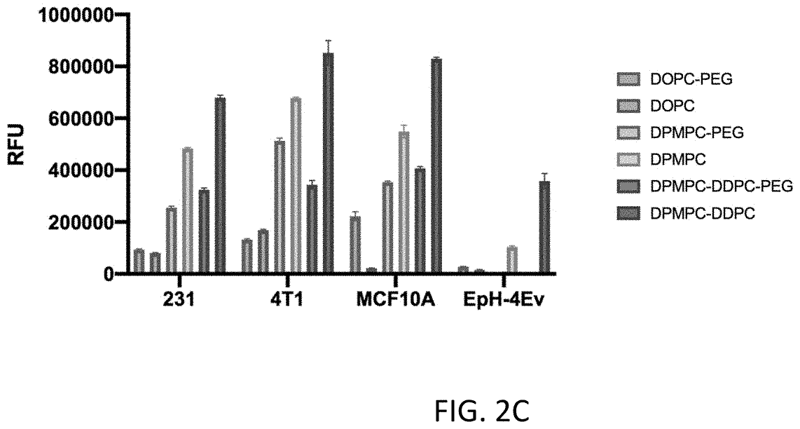

UDLs demonstrated a 25-fold increase in uptake by TNBC cells compared to control DOPC (FIG. 2A-2C). UDLs composed with C7 lipid translocated through the endothelial barrier 2-fold compared to DOPC control (FIG. 3). UDLs composed of C7 and C10 lipid exhibited 2-fold increase in diffusivity in collagen gel compared to DOPC controls.

### Example 2

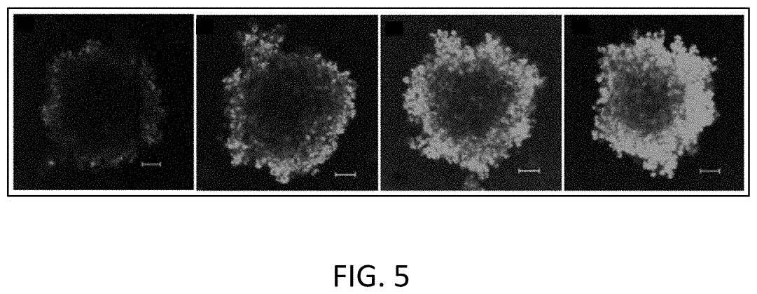

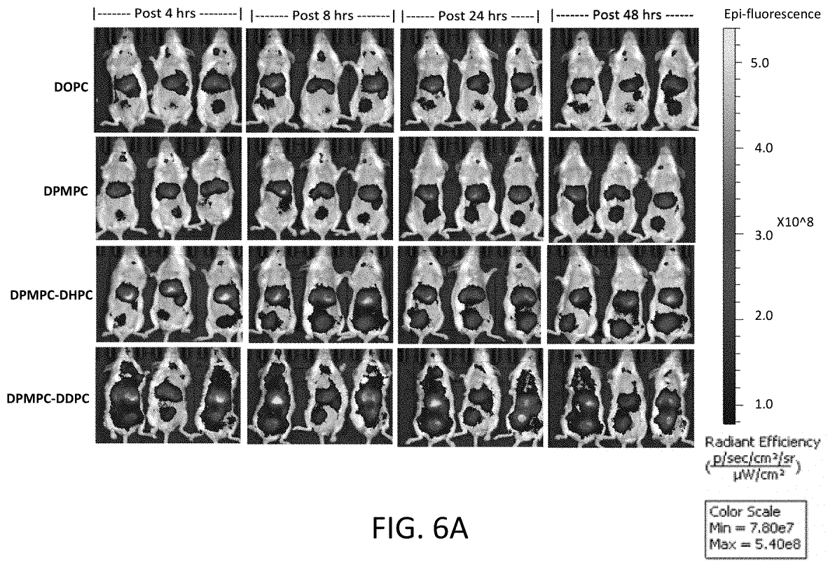

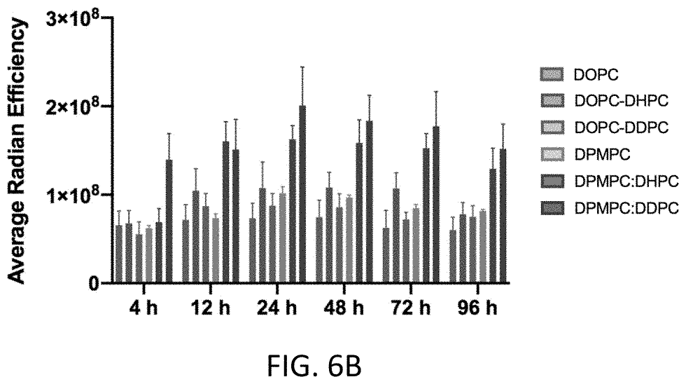

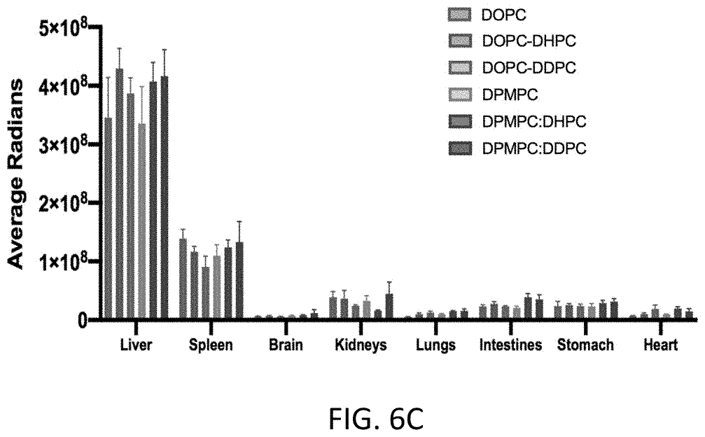

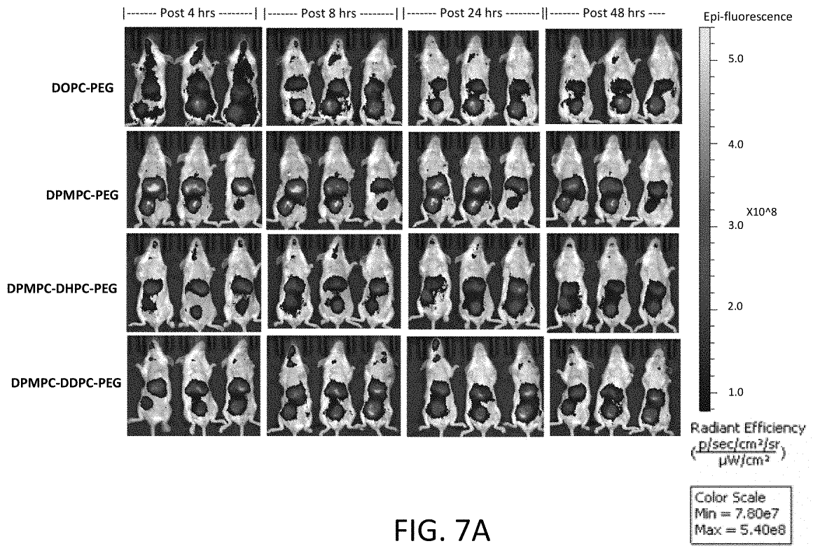

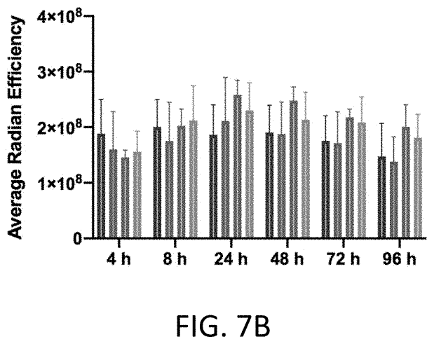

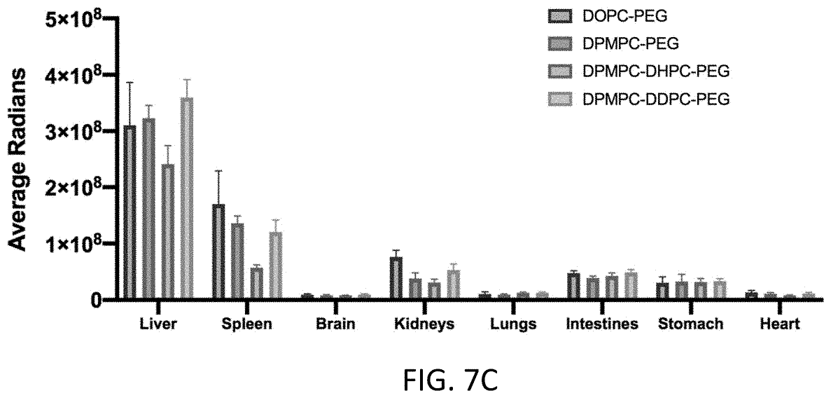

UDLs were formulated with 1,2-dioleoyl-sn-glycero-3-phosphocholine (DOPC) or 1,2-dipalmitoleoyl-sn-glycero-3-phosphocholine (DPMPC) and 1,2-diheptanoyl-sn-glycero-3-phosphocholine (DHPC) or 1,2-didecanoyl-sn-glycero-3-phosphocholine (DDPC) and 1% lipophilic dye, either benzoxazolium, 3-octadecyl-2-[3-(3-octadecyl-2(3H)-benzoxazolylidene)-1-propenyl]-perchlorate (DiO) or 1,1′-dioctadecyl-3,3,3′,3′-tetramethylindotricarbocyanine iodide) (DiR) for visualization in vitro or in vivo, respectively. Liposomes were synthesized using the thin film hydration method. To study the penetration of UDLs, 4T1 spheroids were treated with 400 μM lipid of UDLs for 4 hours at 37° C. and rinsed twice with PBS prior to analysis with confocal microscopy (FIG. 5). For in vivo studies, 6-8-week old female balb/c mice were implanted with subcutaneous orthotopic tumors in the mammary fat pad using 1×10⁶ 4 T1 cells. Tumors were allowed to grow for 2-3 weeks until they reached 200 mm³ in volume. Mice were treated with 10 mg/kg UDL or PEGylated UDL, and imaged using an in vivo imaging system (IVIS) at 4, 8, 24, 48, 72, and 96 h post injection (FIG. 6A and FIG. 7A).

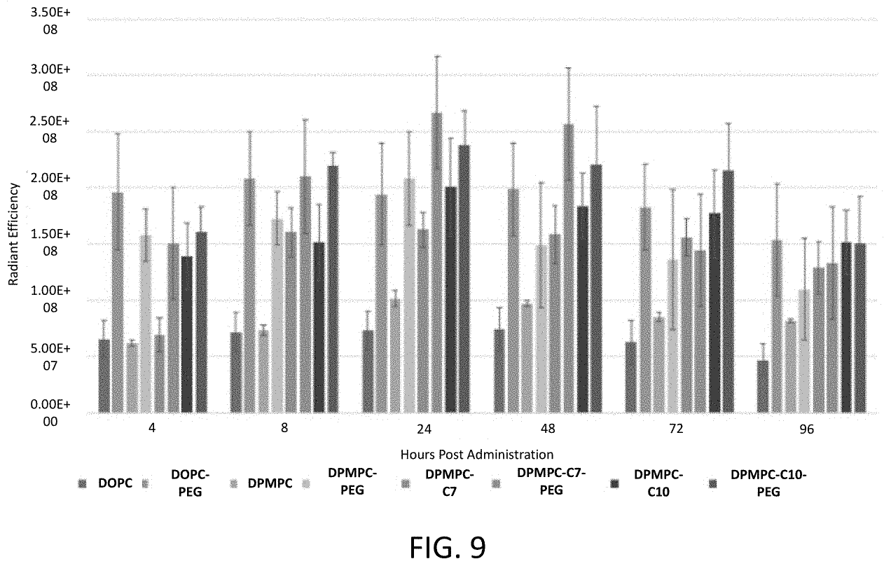

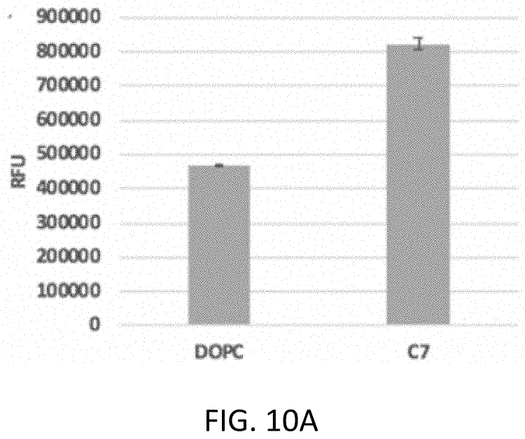

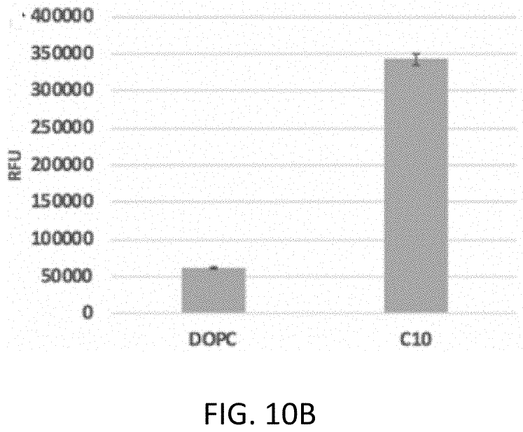

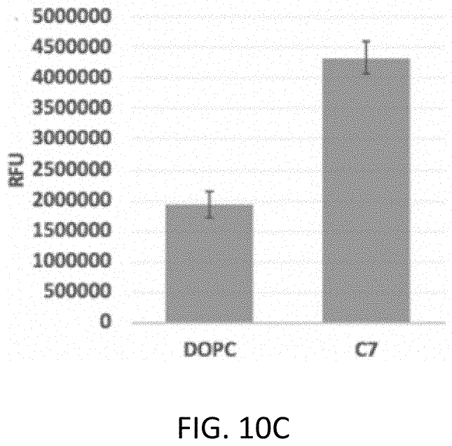

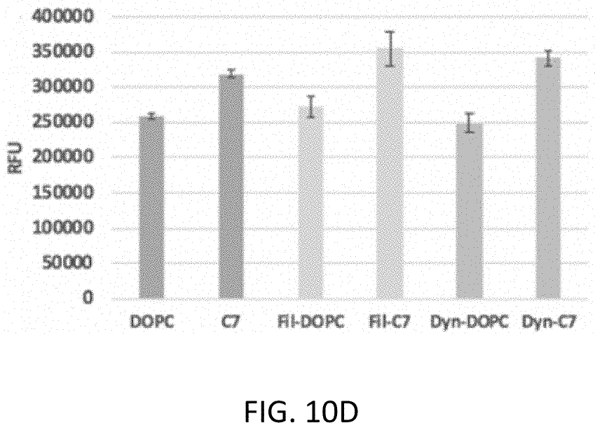

UDLs demonstrated 1.8-6-fold higher penetration in 4T1 spheroids, relative to DPMPC or DOPC control, respectively. In vivo studies demonstrated that DPMPC-DHPC and DPMPC-DDPC UDLs increased tumor accumulation 1.5- and 2-fold relative to controls, respectively. PEGylation of UDL formulations resulted in no statistical differences in tumor accumulation after 72 hours (FIG. 9).

### Example 3

UDLs were formulated with 1,2-dioleoyl-sn-glycero-3-phosphocholine (DOPC), 1,2-dilauroyl-sn-glycero-3-phosphocholine (DLPC; C12) or 1,2-diheptanoyl-sn-glycero-3-phosphocholine (DHPC; C7), and 1% Benzoxazolium, 3-octadecyl-2-[3-(3-octadecyl-2(3H)-benzoxazolylidene)-1-propenyl]-, perchlorate (DiO; a lipophilic carbocyanine dye, which fluoresces when incorporated into a lipid bilayer) for visualization. Liposomes were synthesized using the thin film hydration method. Liposomal size and polydispersity was determined via dynamic light scattering (DLS), using a Brookhaven Zeta-PALS analyzer (Brookhaven Instruments, Holtsville, N.Y., USA).

To study the internalization of UDLs by TNBC cells, MDA-MB-436 or MDA-MB-231 cells were seeded at 75,000 per well in a 24-well plate and incubated overnight at 37° C. Cells were treated with 200 nM UDLs for 2-4 hours at 37° C. Cells were rinsed twice with PBS prior to analysis by flow cytometry (Beckman Coulter, CytoFLEX). Quantification of liposome internalization by cells was determined by DiO fluorescence (excitation/emission: 484 nm), normalizing for background fluorescence (FIGS. 2A, 2C, and 8A). Transendothelial permeability studies were performed using human umbilical vein endothelial cells (HUVECs) seeded on transwell inserts (Corning) and incubated for 2 days prior to use. Then, 200 nM UDLs were added to the upper well of the insert and equal volume of DMEM was added to the bottom of the chamber. Transwells were incubated at 37° C. for 4 hours, then the bottom chamber media was removed and read on a spectrometer for DiO fluorescence.UDL formulations using a combination of DOPC and short chain phospholipids (C7 or C10) were prepared and characterized by size, zeta potential and Young's moduli. UDL-C7 and UDL-C10 formulations demonstrated 1.7-fold and 5.4-fold increase in TNBC cell uptake relative to DOPC control liposomes, respectively. Further, UDL-C7 demonstrated a 2.3-fold increase in transendothelial membrane permeation, compared to DOPC control (FIG. 8C). Preliminary data on the inhibition of cell internalization pathways (clathrin or caveolae) show that UDL-C7 liposomes are internalized via fusion, and not receptor mediated endocytosis.

### Example 4

HELPs were formulated with the following lipid components (Table 1): DOPC; C18:1 (acyl chain length: degree of unsaturation) or 1,2-dipalmitoleoyl-sn-glycero-3-phosphocholine (DPMPC; C16:1), 1,2-didecanoyl-sn-glycero-3-phosphocholine (DDPC; C10:0) and 1,2-diheptanoyl-sn-glycero-3-phosphocholine (DHPC; C7:0). All lipids were purchased from Avanti. A lipophilic carbocyanine dye, benzoxazolium, 3-octadecyl-2-[3-(3-octadecyl-2(3H)-benzoxazolylidene)-1-propenyl]-perchlorate (DiO) was purchased from Biotium. Giant unilamellar vesicles (GUVs) were synthesized using these lipids, with the substitution of 1,2-dioleoyl-sn-glycero-3-phosphoethanolamine-N-(lissamine rhodamine B sulfonyl) (DOPE-Rhodamine) for DiO.

Liposomes were synthesized using the thin film hydration method. Briefly, lipid thin films were synthesized by dissolving all lipid components in chloroform. The lipid mixture was then dried under a rotary evaporator until chloroform was removed. Films were dried overnight in a decanter, followed by hydration in deionized (DI) water. The resulting multilamellar vesicles were extruded 12 times through a 100 nm nanoporous membrane, yielding unilamellar vesicles. Liposomes were then diluted in 10×PBS for a final concentration of 1×PBS (pH 7.4).

Liposome concentration was determined using a phosphate quantification assay. Samples were diluted in 10% sulfuric acid and heated at 200° C. for 1 hour on a hot plate. After cooling briefly, 50 μl of a 30% hydrogen peroxide solution was added, and the mixture was further heated for 45 minutes at 200° C. on a hot plate. Samples were then diluted with 480 μl DI water and transferred to microcentrifuge tubes. A solution of ammonium molybdate (5 mg/ml) and citric acid (20 mg/ml) in DI water was added. Samples were then incubated in a water bath at 45° C. for 25 minutes. Samples were transferred to a 96-well plate and absorbance was measured at 830 nm using a plate reader (Spectra Max). Phosphate concentration was determined using a standard calibration curve of 0-2.5 mM DOPC lipid.

Liposomal size and polydispersity (PDI) were determined via dynamic light scattering (DLS), using a Brookhaven Zeta-PALS analyzer (Brookhaven Instruments, Holtsville, N.Y., USA). Zeta potential of all formulations was determined using phase analysis light scattering (PALS) in 1:100 dilution in PBS. The shape of hyper-elastic and control liposomes was determined using transmission electron microscopy (TEM) at the Electron Microscopy Facility at Harvard Medical School. Briefly, liposomes were incubated for 5 minutes on plasma treated 300-mesh carbon-coated copper grid (Ted Pella Redding, Calif., USA). Uranyl acetate was used to negatively stain the samples for 20 seconds. Grids were then rinsed in triple distilled DI water and dried prior to TEM imaging. Imaging was performed on a JEOL 2100 TEM (JEOL USA Inc, Peabody, Mass., USA) at an accelerating voltage of 100 kV.

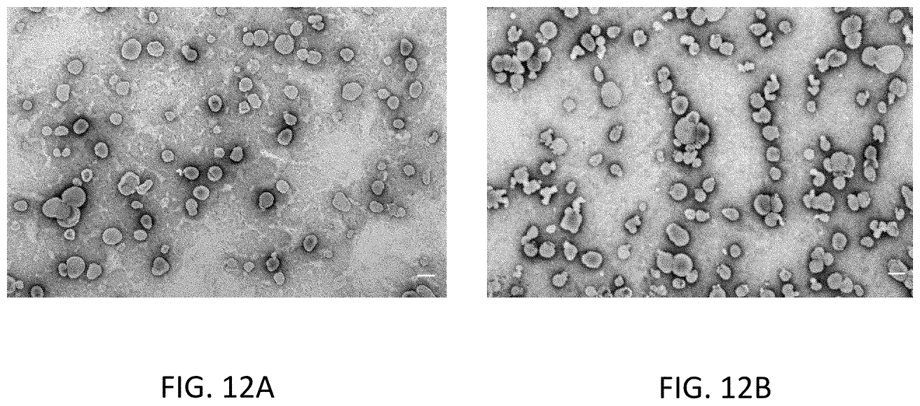

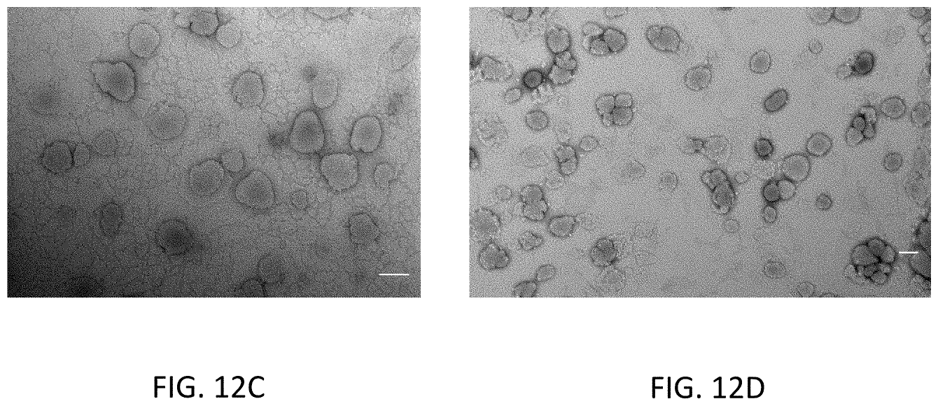

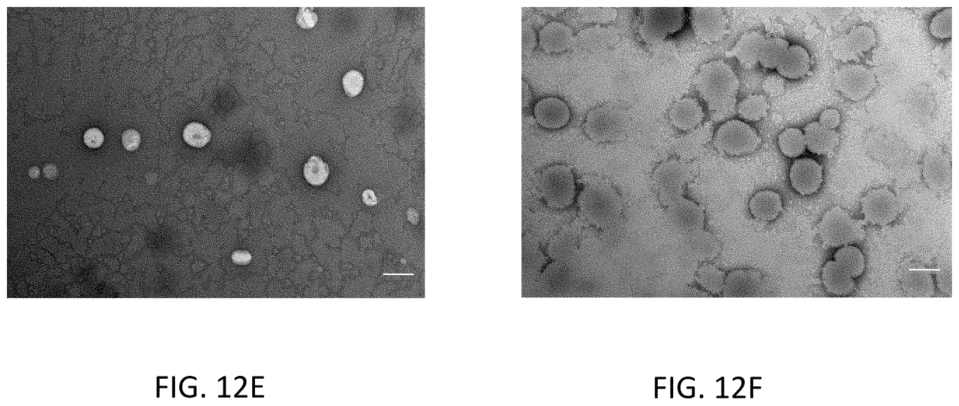

Liposomes were characterized for geometric shape using transmission electron microscopy (TEM) in which all formulations exhibited uniform circular architecture (FIGS. 12A-12F).

### Example 5

Cellular Viability: The cytotoxicity of non-drug loaded liposomes was studied using human and murine neoplastic cells MDA-MB-231 and 4T1, respectively. Cytotoxicity was also evaluated using human and murine non-neoplastic control cells MCF10A and EpH-4Ev, respectively. Cells were seeded at 10,000 per well in a 96-well tissue culture plate and incubated overnight at 37° C. Cells were treated with 200 lipid of HELPs, control liposomes, or PBS and incubated for 24 h at 37° C. Alamar blue (Fisher) was diluted 10-fold and applied to cells and incubated for 4 h at 37° C. Fluorescence at 570/590 excitation emission was evaluated using a plate reader (SpectraMax i3 fluorescence spectrophotometer; Molecular Devices Corp, Sunnyvale, Calif., USA) and viability was determined relative to PBS treatment.

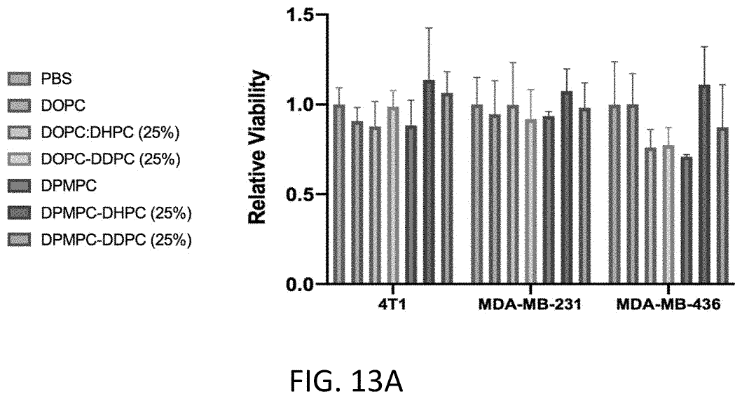

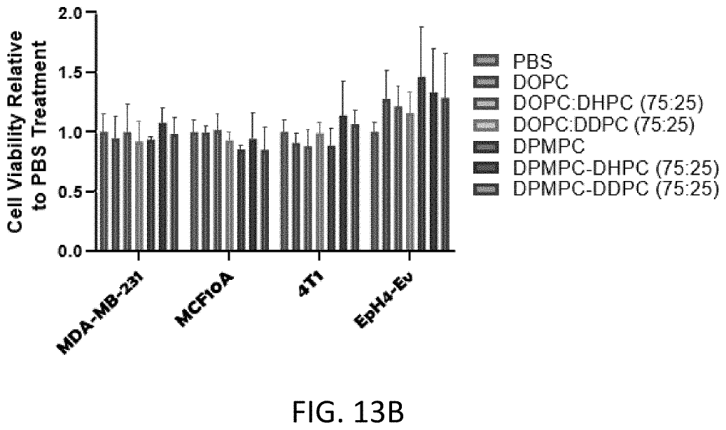

This assay quantitatively determines the metabolic activity of cells by measuring their ability to reduce resazurin. Resazurin is a cell-permeable and non-toxic reagent which has very low fluorescence. Within living cells, resazurin is reduced to resorufin during aerobic respiration within the mitochondria. The resulting resorufin is a highly fluorescent compound which is bright red in color. Treatment with a given liposome formulation can be quantitatively compared to control treatment with PBS to determine effect on cell viability. This experiment demonstrated that there was no significant effect on cell viability from hyper-elastic or control liposomes treatment in neoplastic or non-neoplastic cell lines, relative to treatment with PBS (FIGS. 13A-13B).

### Example 6

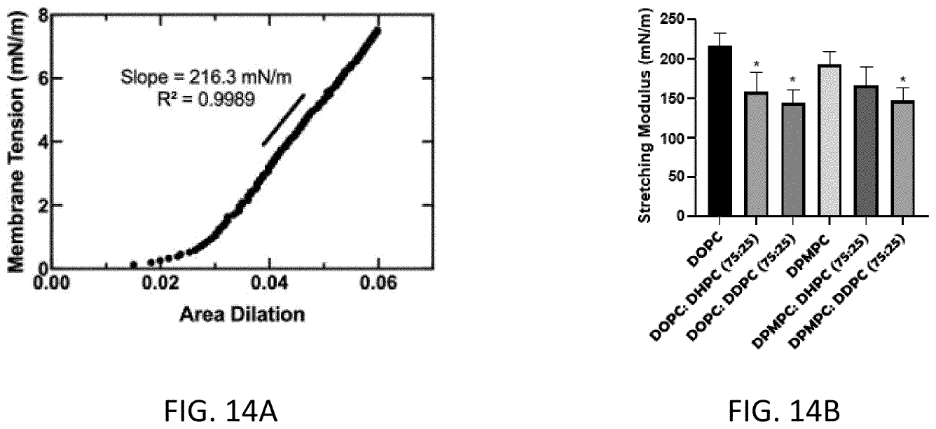

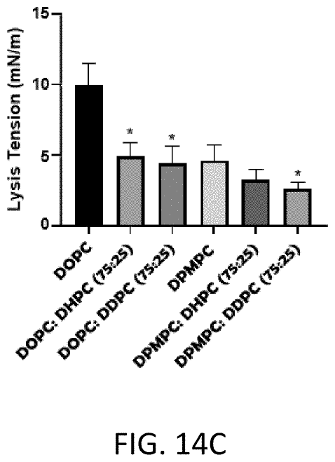

Mechanical Characterization: Mechanical measurements of the lipid bilayer were obtained using micropipette aspiration (MPA). This measurement stretches the lipid membrane to the point of irreversible deformation (lysis). A tension versus areal strain curve can be obtained from these experiments, in which the slope of the linear region of this curve corresponds to the stretching modulus (FIG. 14A). Control UDLs composed of 100 mol % DOPC or DPMPC exhibited the highest stretching moduli of 216±16.2 and 193±16.9 mN/m, respectively (FIG. 14B).

Hyper-elastic UDLs formulated with 75 mol % DOPC with 25 mol % DHPC or DDPC exhibited significantly lower stretching moduli of 157±24.1 and 142±15.3 mN/m, respectively, relative to 100 mol % DOPC liposomes. Hyper-elastic UDLs formulated with 75 mol % DPMPC and 25 mol % DHPC or DDPC also exhibited lower stretching moduli of 166±23.1 and 147±16.6 mN/m, respectively. However, only the DPMPC-DDPC formulation had a significantly lower stretching modulus, relative to 100 mol % DPMPC liposomes (Table 3).

TABLE 3
Mechanical characterization of control and ultra-deformable liposomes.

| | Concentration | Stretching Modulus | Lysis Tension |
|---|---|---|---|
| Sample | (mol:mol) | (mN/m) | (mN/m) |
| DOPC | 100 | 216 | 10 |
| DOPC:DHPC | 75:25 | 157 | 4.9 |
| DOPC:DDPC | 75:25 | 142 | 4.5 |
| DPMPC | 100 | 193 | 4.6 |
| DPMPC:DHPC | 75:25 | 166 | 3.3 |
| DPMPC:DDPC | 75:25 | 147 | 2.6 |

The lysis tension of HELPs, which indicated the pressure at which the HELP was irreversibly deformed and the membrane ruptured was measured. A high lysis tension indicated a bilayer which required greater force to induce membrane rupture, indicating a stiffer vehicle. HELPs composed of 100 mol % DOPC or DPMPC had the highest lysis tension, indicating it took the most pressure to rupture these membranes. Addition of 25 mol % DHPC or DDPC to 75 mol % DOPC HELPs resulted in a significant reduction in the lysis tension of 4.9±0.9 and 4.5±1.2 mN/m, respectively (FIG. 14C). Significantly lower lysis tension of DPMPC based hyper-elastic HELPs was also noted, in which the addition of 25 mol % DDPC yielded vesicles with lysis tension of 2.6±0.5 mN/m.

### Example 7

Cellular Internalization and Binding: All cell lines were purchased from ATCC (Manassas, Va.). Dulbecco's modified Eagle's medium (DMEM) was purchased from Gibco and RPMI-1640 medium was purchased form ATCC (Manassas, Va.). Media supplements, including penicillin-streptomycin (pen-strep), fetal bovine serum (FBS), and fibronectin were purchased from ThermoFisher. Transwells of 0.4-3 μm pore size, 24-well transwell companion plates, and 96-well ultra-low attachment micro-spheroid plates were purchased from Corning.

The cellular internalization of HELPs incorporating 5-35% DHPC or DDPC was determined quantitatively using flow cytometry. The cellular internalization of HELPs was studied using human and murine triple negative breast cancer (TNBC) cell lines MDA-MB-231 and 4T1, respectively. Human and murine nonneoplastic mammary epithelial cell lines, MCF10A and murine EpH4-Ev, respectively, were also utilized for internalization studies as controls. Cells were seeded at 50,000 per well in a 24-well tissue culture plate and incubated overnight at 37° C. Cells were then treated with 200 μM lipid of HELPs, control liposomes, or PBS for 4 hours at 37° C.

Cells were then rinsed twice with PBS, trypsinized and collected. Trypsin was neutralized via the addition of equal parts complete media followed by centrifugation. Cells were resuspended in PBS and rinsed via centrifugation. Quantitative analysis of internalization was performed using flow cytometry (Beckman Coulter, CytoFLEX) in which measurement of 5,000 cells per condition were obtained. Quantification of liposome internalization by cells was determined by DiO fluorescence (ex/em: 484 nm), normalizing for background fluorescence. For binding studies, the same protocol was executed, except cells were incubated at 4° C. instead of 37° C. for 4 h after the addition of 200 μM liposomes.

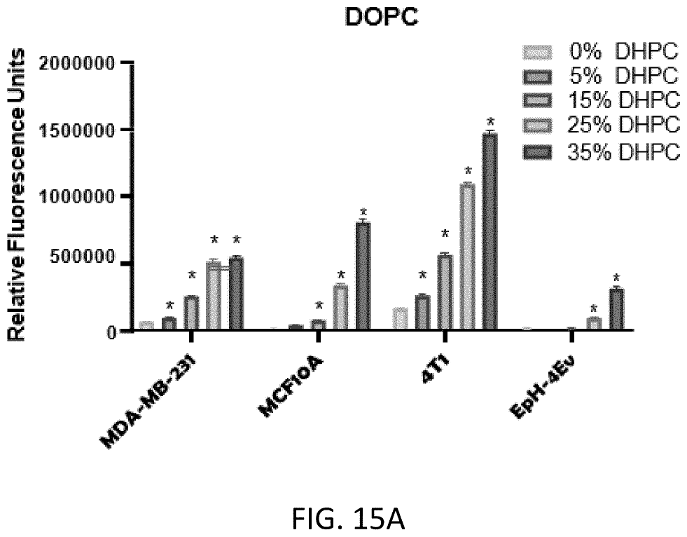

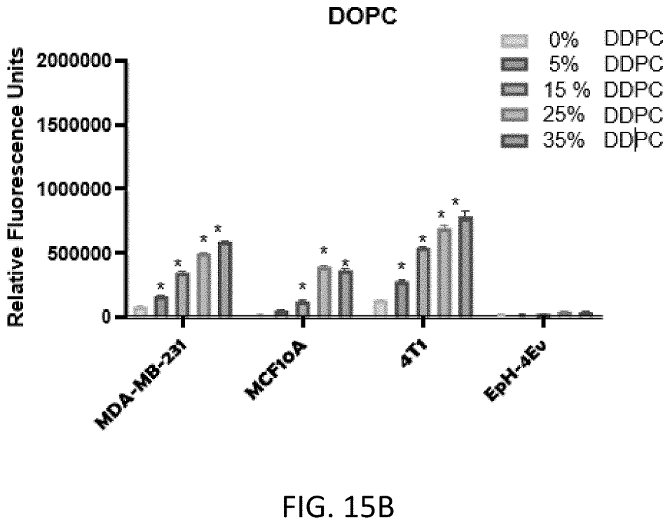

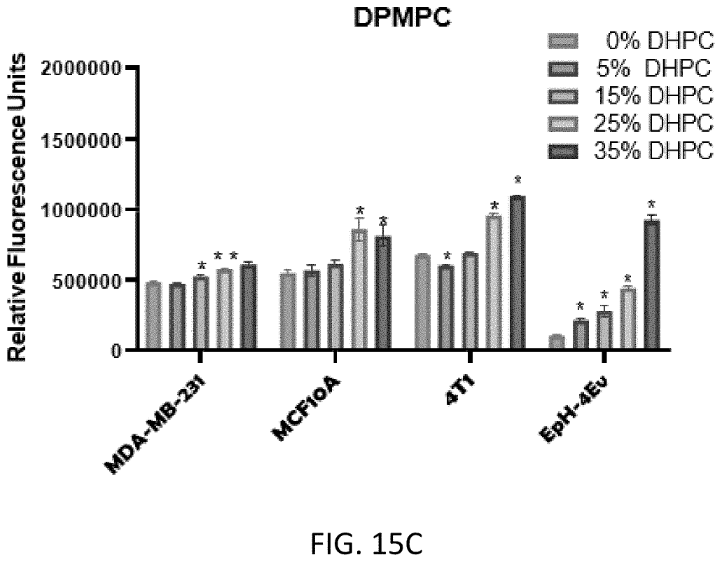

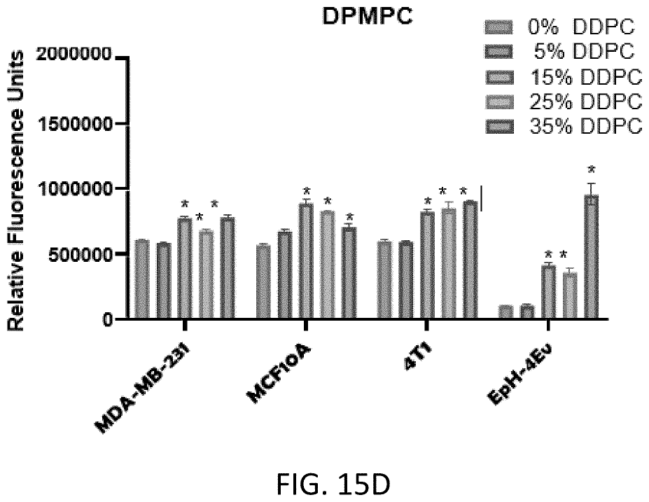

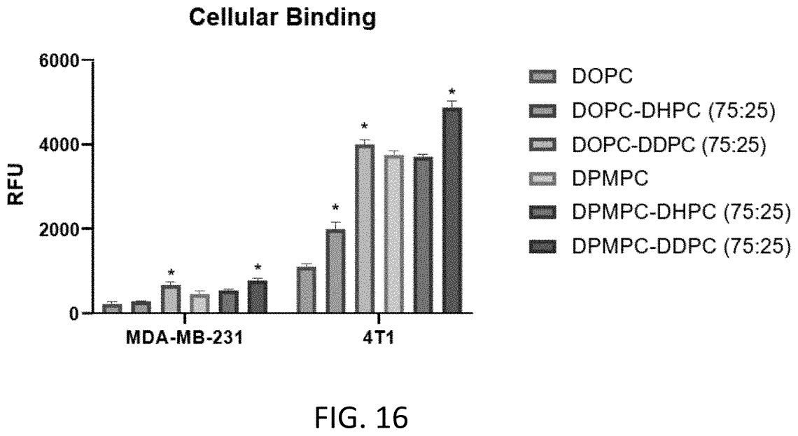

A significant, increase in internalization by both TNBC and nonneoplastic controls was observed with incorporation of 5 to 35 mol % of DHPC or DDPC in combination with DOPC (FIGS. 15A and 15B). Incorporation of DHPC or DDPC in combination with DPMPC demonstrated higher uptake in cancerous cells for formulations of 25-35 mol % and 15-35 mol %, respectively (FIGS. 15C and 15D) HELPs demonstrated equivalent or higher cellular internalization in TNBC cells compared to healthy epithelial cells. In murine cells, incorporation of 25 mol % DHPC or DDPC was identified as having the highest cellular internalization and selective uptake in TNBC cells relative to control cells. Thus 25 mol % DHPC or DDPC liposomes were utilized for further investigation. The cellular binding of HELPs in TNBC cells was evaluated. Cellular binding of HELPs with 25 mol % DDPC was significantly higher relative to control liposomes in TNBC cells (FIG. 16). Overall, however, the cellular binding was roughly 250-fold lower than that of cellular internalization for MDA-MB-231 and 4T1 cells, respectively. These data indicate that HELPs are internalized by energy dependent mechanisms, which is in contrast to energy independent processes, such as fusion, exhibited by other soft nanoparticles.

### Example 8

Transendothelial Permeability and HUVEC Internalization: Transwell inserts with pore size 3 μm were coated with 100 μg/ml fibronectin for 1.5 hours at 37° C. prior to use. Human umbilical vein endothelial cells (HUVECs) passage 2-6, were seeded at 20,000 per transwell insert and incubated for 48 hours at 37° C. Following incubation, transepithelial electrical resistance (TEER) measurements were acquired and analyzed to determine that HUVECs had formed an intact monolayer. Once a uniform monolayer was achieved, transwells were incubated with murine TNBC MDA-MB-231 conditioned medium for 24 hours to induce permeability. Conditioned medium was obtained from a flask of MDA-MB-231 cells which had been cultured to confluency. Media was removed and centrifuged at 200×g for 5 minutes, prior to addition to transwells. Then, 200 μM lipid of HELPs and control liposomes diluted in DMEM were added to the top of the transwell insert and 600 μl of DMEM was added to the bottom of the transwell. After incubating for 4 hours, the media in the bottom chamber was collected and measured for DiO fluorescence using a plate reader (SpectraMax i3 fluorescence spectrophotometer; Molecular Devices Corp, Sunnyvale, Calif., USA) at excitation/emission wavelengths of 484/501 nm. The relative transendothelial particle number was calculated as a percent of the total initial particles, using the following equation:

$$\% Liposome Translocation = \frac{Fluorescence of bottom well medium}{Fluorescence of liposome stock in medium} \times 100$$

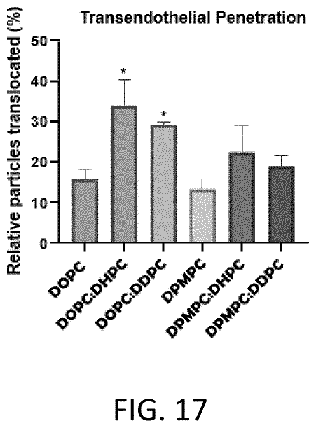

The integrity of the HUVEC monolayer was characterized by measuring the transendothelial electrical resistance (TEER) across the cell layer using an epithelial voltommeter (World Precision Instruments). HUVEC seeded transwells with TEER values of 10 Ω·cm² were used, which indicated an intact monolayer. Liposomes composed of 75 mol % DOPC and 25 mol % DHPC or DDPC demonstrated significantly higher transendothelial penetration relative to 100 mol % DOPC liposomes, increasing translocation of liposomes 2.2 and 1.9-fold, respectively (FIG. 17). DPMPC liposomes incorporating 25 mol % DHPC or DDPC exhibited 1.7 and 1.4-fold higher transendothelial penetration relative to control liposomes, respectively.

### Example 9

Spheroid Accumulation and Penetration: TNBC spheroids were cultured by seeding 1,000 4T1 cells in each well of an ultra-low attachment 96 well plate (Corning). Spheroids were treated with 400 μM lipid of hyper-elastic or control liposomes for 4 h at 37° C. After incubation, spheroids were rinsed twice with sterile PBS for 15 minutes and fixed with 4% paraformaldehyde for 30 minutes at room temperature. Spheroid images were acquired using a confocal laser scanning microscope (CLSM) at 20× magnification. Hyper-elastic and control liposome penetration within spheroids was determined by quantifying DiO fluorescence at the center of the spheroid. Image analysis was performed using ImageJ. Using the line tool in ImageJ, the fluorescence across each spheroid image was quantified using eight evenly spaced lines. Each line spanned the full spheroid diameter, thereby sectioning the spheroid into 16 even segments. The diameter distance vs fluorescence (x,y) data was exported from ImageJ. Based on the total diameter length, each data set was segmented into 10 sections of equal length. Each section was then graphed in GraphPad Prism and the area under the curve (AUC) of each section was quantified. For presenting data in terms of radial penetration, the AUC of the outer most sections were added together, corresponding to fluorescence present in 0-20% of the total spheroid radius. This analysis was repeated for the adjacent spheroid sections, which corresponded to 20-40% of the total spheroid radius. This process was repeated for remaining sections of 40-60%, 60-80%, and 80-100% of the total spheroid radius. Spheroid accumulation was determined by quantifying the area under the curve of DiO intensity vs diameter plots. Background fluorescence was determined using a non-treated spheroid sample.

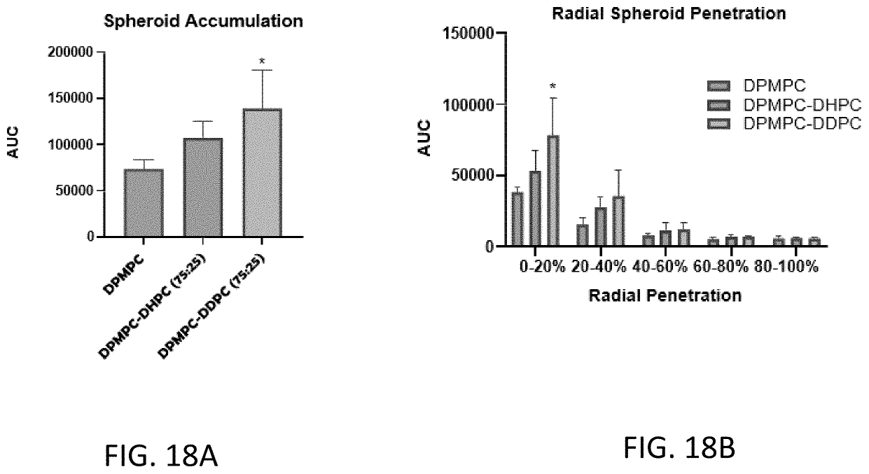

The increased depth of penetration of hyper-elastic liposomes in 3D spheroids (FIGS. 5, 18A, and 18B) indicated the ability of these liposomes to reversibly deform while squeezing between cell gaps and pores of a dense cellular matrix. Relative to control DPMPC liposomes, a 1.4- and 2-fold increase in spheroid penetration was observed at radial spheroid depths of up to 15 μm for DPMPC-DHPC (75:25 mol:mol) and DPMPC-DDPC (75:25 mol:mol), respectively. At radial depths of up to 15 μm, an increasing trend of fold differences was still observed for DPMPC-DHPC (75:25 mol:mol) and DPMPC-DDPC (75:25 mol:mol) of 1.8- and 2.3-fold higher penetration, respectively, relative to DPMPC 100 mol % control liposomes.

Radial penetration of DPMPC based liposomes in TNBC spheroids in which average spheroid radius was approximately 75 μm. Radial spheroid penetration is denoted in % of total radius, in which 0-20% indicates the outer radius and 80-100% indicates the inner radius. A significant increase in radial penetration was noted in at radial distances of 0-20% equivalent to 15 μm.

### Example 10

Tumor Accumulation and Biodistribution: All animal studies were performed in accordance with protocols approved by the Institutional Animal Care and Use Committee (IACUC) at Northeastern University. A syngeneic orthotopic tumor model was constructed using 6-8 week old female balb/c mice purchased from Charles River. Tumors were implanted in the mammary fat pad using 1×10⁶ 4 T1 cells. Tumors were measured daily with calipers until they reached approximately 200 mm³ in volume. The volume of each tumor was calculated using the following formula: V=0.5×L×W², in which L was the longest dimension and W was the dimension perpendicular to L. Mice were randomized into 6 treatment groups (n=3-4) and administered with 10 mg lipid of liposomes per kg mouse mass (10 mg/kg) via systemic injection. Tumor accumulation was monitored using an in vivo imaging system (IVIS) Lumina II (DiR ex/em: 748/780) at time points 4, 8, 24, 48, 72, and 96 h post injection.

Biodistribution was determined at the end of the study, 96 hours post liposome injection. Mice were euthanized and the brain, heart, liver, lung, kidney, stomach, spleen, and tumors were excised. Ex vivo DiR fluorescence in organs and tumors was determined using an IVIS Lumina II.

To determine the effect of liposomal elasticity on tumor accumulation and biodistribution, a syngeneic TNBC model was utilized. DiR, a near infrared (NIR) lipophilic dye, was incorporated in all liposomal formulations to enable in vivo imaging of liposome tumor accumulation in real-time for 4-96 hours after systemic administration (FIG. 6A).

HELPs composed of DPMPC-DHPC (75:25 mol:mol) and DPMPC-DDPC (75:25 mol:mol) accumulated in murine TNBC tumors significantly more than control DPMPC liposomes at time points 8, 48, 72 and 96 h post administration (FIG. 6B). At 24 h post administration, tumor accumulation of DPMPC-DHPC (75:25 mol:mol) and DPMPC-DDPC (75:25 mol:mol) was 1.6- and 2-fold higher than that of 100 mol % DPMPC control liposomes. In contrast to in vitro cell uptake and transendothelial penetration data, DOPC-DHPC (75:25 mol:mol) and DOPC-DDPC (75:25 mol:mol) HELP formulations did not increase tumor accumulation relative to DOPC (100 mol %) control liposomes, with the single exception of DOPC-DHPC (75:25 mol:mol) at the 72 h time point. This may be due to complexities of liposomal behavior in vivo, in which factors like blood circulation time play a critical role in determining tumor accumulation.

Biodistribution of hyper-elastic and control liposomes demonstrated no significant differences in accumulation within the liver, spleen, kidneys, brains, or stomach (FIG. 6C). Accumulation within the liver and spleen was the highest of all organs, as anticipated, because these organs are the predominant sites of liposome clearance from the blood.

### Example 11

Liposomal Serum Adsorption: Adsorption of bovine serum albumin (BSA) was quantified using a spectrofluorometer-based assay. Liposomes were added to a solution of 10 μM BSA in 10 serial additions, for final concentrations of 0-400 μM liposome and 5 μM BSA. Samples were measured using a spectrofluorometer (Photon Technology International QuantaMaster) at an excitation of 280 nm. Fluorescence emission spectra were recorded at 300-350 nm. BSA quenching was calculated by determining the Stern-Volmer quenching constant using the equation below:

$$\frac{Fo}{F} = 1 + Ksv [ Q ]$$

where Fo and F are the steady state fluorescence intensities in absence and presence of liposomes (quencher), respectively, and Kₛᵥ is the Stern-Volmer quenching constant, which denotes the amount of BSA associated with the liposome surface.

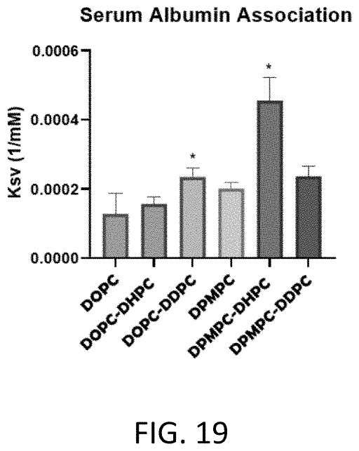

In order to quantify the association of BSA with hyper-elastic and control liposomes, the Stern-Volmer (Kₛᵥ) association constant of BSA with hyper-elastic and control liposomes was determined. A higher Kₛᵥ indicates that more BSA is associated with the liposome's surface. DPMPC-DHPC (75:25 mol:mol) and DOPC-DDPC (75:25 mol:mol) liposomes exhibited a significant increase in Kₛᵥ relative to 100 mol % DOPC or DPMPC liposomes (FIG. 19).

### Example 12

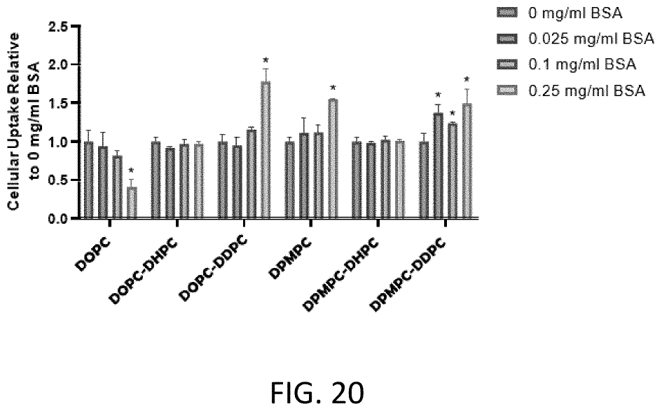

BSA Association Effect on Liposome Uptake: The effect of pre-incubation of liposomes with varying concentrations of BSA on cellular internalization was studied. Murine 4T1 cells were seeded at 50,000 cells per well and allowed to attach overnight. Liposomes (200 μM lipid) were pre-incubated with 0-0.25 mg/ml BSA for 1 h at 37° C. Liposomes were then diluted in medium without FBS, added to cells, and incubated for 4 h at 37° C. Internalization of BSA-incubated liposomes was determined quantitatively using flow cytometry (FIG. 20).

### Example 13

Polyethylene Glycol (PEG) Liposome Synthesis and Quantification: For the addition of PEG to liposome formulations, 1,2-dipalmitoyl-sn-glycero-3- phosphoethanolamine-N-[methoxy(polyethylene glycol)-2000] (DPPE-PEG₂₀₀₀) lipid was purchased from Avanti.

PEGylated HELPs were formulated with the addition of 5 mol % 1,2-dipalmitoyl-sn-glycero-3-phosphoethanolamine-N-[methoxy(polyethylene glycol)-2000] (DPPE-PEG₂₀₀₀) in each of the following formulations: DOPC: DPPE-PEG₂₀₀₀ (95:5 mol:mol), DPMPC-DPPE-PEG₂₀₀₀ (95:5 mol:mol), DPMPC-DHPC-DPPE-PEG₂₀₀₀ (70:25:5 mol:mol) and DPMPC-DDPC-DPPE-PEG₂₀₀₀ (70:25:5 mol:mol). Thin film hydration followed by extrusion was used to synthesize and characterize liposomes, as detailed in Example 4.

### Example 14

Cellular Internalization of PEGylated Liposomes: Cellular internalization of PEGylated liposomes was performed using methods of Example 7. Cellular internalization was quantified using flow cytometry in which data were normalized to cellular fluorescence with PBS treatment.

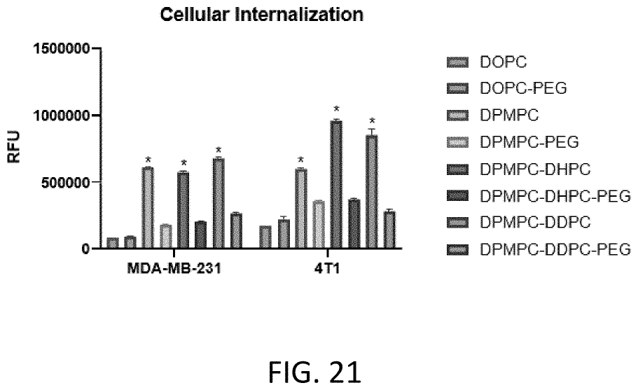

The effect of PEGylation on cellular internalization of hyper-elastic and control liposomes using human and murine TNBC cells was determined (FIG. 21). The cellular internalization of DPMPC-DHPC-DPPE-PEG2000 (70:25:5 mol:mol) and DPMPC-DDPC-DPPE-PEG (70:25:5 mol:mol) liposomes was reduced 2.8- and 3-fold, relative to their non-PEGylated counterparts, respectively. However, for PEGylated DOPC liposomes, there was no significant difference in cellular internalization relative to non-PEGylated DOPC liposomes. In contrast, PEGylation of DPMPC control liposomes resulted in a 1.7- to 3.4-fold reduction in cellular internalization by human and murine TNBC cells, respectively. PEGylation of nanoparticles has been demonstrated to reduce nanoparticle-cell interactions due to steric hindrance caused by PEG153. The data generally support this principle, with the exception of PEGylated DOPC liposomes. It is interesting to note that though structurally DPMPC differs from DOPC only by the absence of two carbon atoms in its acyl chains, these two control formulations exhibit different patterns of cellular internalization upon PEGylation.

### Example 15

Spheroid Accumulation and Penetration of PEGylated Liposomes: Accumulation and penetration of PEGylated liposomes was evaluated using the 4T1 spheroid model detailed in Example 9. ImageJ analysis was used to quantify radial penetration of PEGylated liposomes.

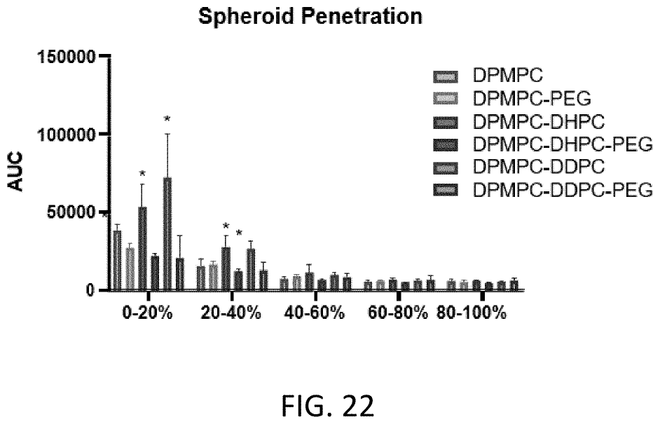

Tumor spheroid penetration and accumulation studies revealed that PEGylations of liposomes had a detrimental impact on tissue penetration (FIG. 22). DPMPC-DDPC-DPPE-PEG₂₀₀₀ (70:25:5 mol:mol) exhibited a 3.5-fold reduction in liposome penetration at a radial distance of 0-20% (corresponding to lapproximately 15 μm), relative to non-PEGylated DPMPC-DDPC (75:25 mol:mol). Penetration of DPMPC-DHPC-DPPE-PEG₂₀₀₀ (70:25:5 mol:mol) and DPMPC-DDPC-DPPE-PEG₂₀₀₀ (70:25:5 mol:mol) was 2.2- and 2-fold lower than their non-PEGylated counterparts at radial distances of up to 30 μm, respectively.

### Example 16

Effect of BSA Adsorption on Cellular Internalization of PEGylated Liposomes: Pre-incubation of PEGylated liposomes with BSA prior to treatment of 4T1 cells was performed using methods detailed in Example 11. Cellular internalization was quantified using flow cytometry, in which data was normalized to cellular fluorescence with PBS treatment.

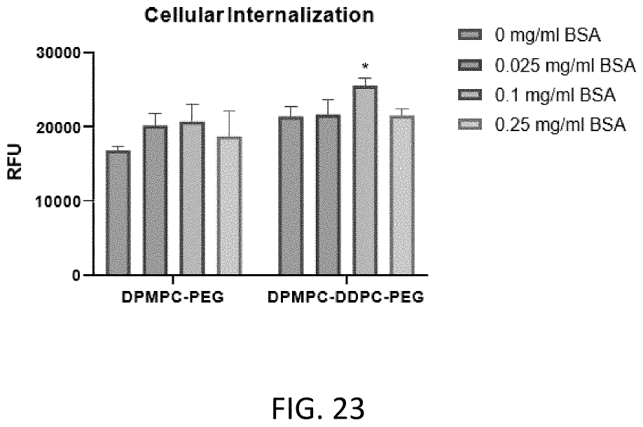

The effect of BSA pre-incubation on the cellular internalization of PEGylated liposomes was evaluated. Using DPMPC-PEG₂₀₀₀ (95:5 mol:mol) and DPMPC-DDPC-DPPE-PEG₀₀(70:25:5 mol:mol) liposomes that were pre-incubated with 0-0.25 mg/ml of BSA. Overall, the BSA incubation with PEGylated liposomes had little effect on their cellular internalization (FIG. 23). A moderate increase in the cellular internalization of DPMPC-DDPC-DPPE-PEG₂₀₀₀ (70:25:5 mol:mol) after incubation with 0.1 mg/ml BSA incubation was observed.

### Example 17

Tumor Accumulation and Biodistribution of PEGylated HELPs in TNBC Tumor Model: A syngeneic orthotopic tumor model was used, as detailed in Example 10. Tumor accumulation was monitored using an in vivo imaging system (IVIS) Lumina II (ex/em: 748/780) at time points 4, 8, 24, 48, 72, and 96 h post injection. The biodistribution of PEGylated liposomes was determined at the end point of the study.

To understand the effect of liposome PEGylation on tumor accumulation in vivo, the murine 4T1 TNBC tumor model discussed in Example 10 was utilized. PEGylated liposomes were administered systemically at 10 mg/kg (lipid mass/mouse mass) and monitored for tumor accumulation and biodistribution for 4-96 hours (FIG. 7A). PEGylation of HELP formulations demonstrated no statistical differences in tumor accumulation at any time point of the study, relative to control liposomes (FIG. 7B).

The biodistribution of PEGylated liposomes was statistically equivalent in all organs, except the lungs and spleen. In the lungs, DPMPC-DHPC-DPPE-PEG₂₀₀₀ (70:25:5 mol:mol) exhibited higher accumulation relative to DPMPC-DPPE-PEG₂₀₀₀ (95:5 mol:mol) (FIG. 7C). However, in the spleen DPMPC-DHPC-DPPE-PEG₂₀₀₀ (70:25:5 mol:mol) accumulated significantly less than control DPMPC-DPPE-PEG₂₀₀₀ (95:5 mol:mol) liposomes. This may indicate the ability of DPMPC-DHPC-DPPE-PEG₂₀₀₀ (70:25:5 mol:mol) to avoid uptake by the lymphatic system relative to other PEGylated liposomes tested herein.

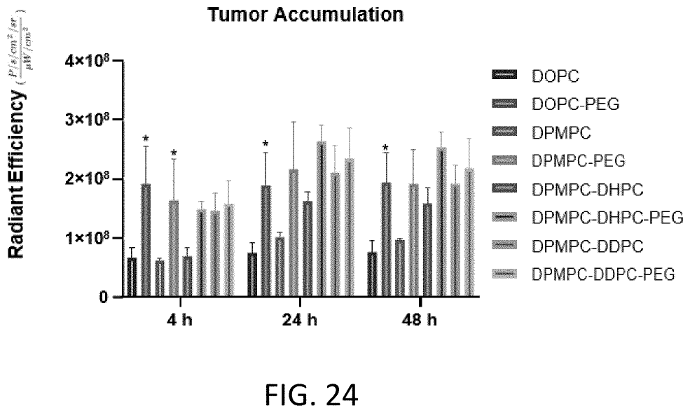

With the addition of PEG, control liposomes composed of DOPC or DPMPC demonstrated increased tumor accumulation. However, PEGylated HELP liposomes exhibited statistically equivalent tumor accumulation of relative to non-PEGylated DPMPC-DHPC (75:25 mol:mol) and DPMPC-DDPC (75:25 mol:mol) (FIG. 24). Further, tumor accumulation of DPMPC-DHPC (75:25 mol:mol) and DPMPC-DDPC (75:25 mol:mol) was equivalent to that of PEGylated DOPC, which is a model system for liposomal drug delivery.

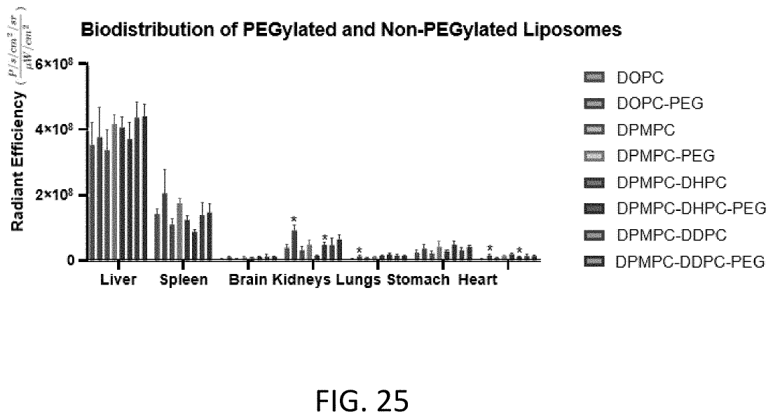

Upon comparing the biodistribution of PEGylated and non-PEGylated liposomes (FIG. 25), a significant increase in kidney accumulation was noted for DPMPC-DHPC-DPPE-PEG2000 (70:25:5 mol:mol) relative to its non-PEGylated counterpart. PEGylation of DOPC control liposomes resulted in a significant increase in kidney, lung, and heart accumulation. In the heart, a significant reduction in the accumulation of DPMPC-DHPC-DPPE-PEG2000 (70:25:5 mol:mol) relative to non-PEGylated DPMPC-DHPC(75:25 mol:mol) was also observed. Overall, these data suggest that PEGylation may increase uptake in the kidneys and lungs, while reducing uptake of PEGylated HELPs in the heart. Further investigation into the role of PEG in regulating in vivo biodistribution would be necessary to discern the mechanisms by which uptake in specific organs may be occurring.

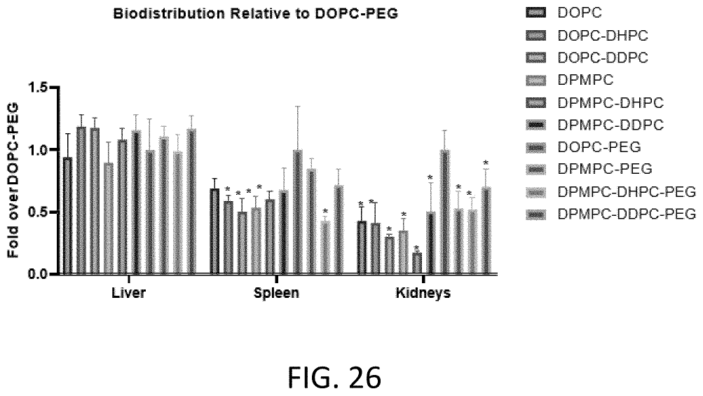

When comparing the biodistribution of non-PEGylated liposomes relative to DOPC-DPPE-PEG₂₀₀₀ (95:5 mol:mol) liposomes, it was noted that non-PEGylated HELPs accumulated significantly less in the kidneys and spleen (FIG. 26). This indicates that without the use of PEG, novel HELP formulations may evade clearance within the renal and lymphatic systems, thereby pro-longing blood circulation, relative to conventionally utilized DOPC-PEG (95:5 mol:mol) liposomes.

The foregoing Examples 1-3 have been described in “Debra Auguste and Danielle Large, 240c—Ultra-Deformable Liposomes for Enhanced Drug Delivery, presented at: AIChE annual meeting, Nov. 16-20, 2020, virtual,” and “Debra Auguste and Danielle Large—Ultra-Deformable Liposomes for Enhanced Drug Delivery, presented at: BMES, Oct. 6-9, 2021, virtual,” the entireties of which are incorporated herein by reference.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed:

1. A liposome comprising a first lipid and a second lipid, wherein:
the first lipid comprises a first hydrophilic head linked to a first aliphatic tail;
the second lipid comprises a second hydrophilic head linked to a second aliphatic tail having at least two carbons less than the first aliphatic tail; and
the liposome is ultra-deformable.

2. The liposome of claim 1, wherein the first hydrophilic head and the second hydrophilic head are the same.

3. The liposome of claim 2, wherein the first and second hydrophilic heads each comprise phosphocholine, phosphoethanolamine, phosphoinosine or phosphoserine.

4. The liposome of claim 3, wherein the first and second hydrophilic heads each comprise phosphocholine.

5. The liposome of claim 1, wherein the first aliphatic tail is unsaturated, and has from 13 carbon atoms to 35 carbon atoms in a linear arrangement.

6. The liposome of claim 5, wherein the first aliphatic tail is unsaturated and has from 13 carbon atoms to 21 carbon atoms in a linear arrangement.

7. The liposome of claim 1, wherein the first lipid is 1,2-dipalmitoleoyl-sn-glycero-3-phosphocholine, 1,2-dimyristoyl-sn-glycero-3-phosphocholine, 1,2-distearoyl-sn-glycero-3-phosphocholine, 1,2-dioleoyl-sn-glycero-3-phosphoethanolamine, 1,2-dipalmitoyl-sn-glycero-3-phospho-(1′-rac-glycerol), 1,2-distearoyl-sn-glycero-3-phosphoethanolamine, 1,2-distearoyl-sn-glycero-3-phospho-(1′-rac-glycerol), or 1,2-dioleoyl-sn-glycero-3-phosphocholine.

8. The liposome of claim 7, wherein the first lipid is 1,2-dipalmitoleoyl-sn-glycero-3-phosphocholine or 1,2-dioleoyl-sn-glycero-3-phosphocholine.

9. The liposome of claim 1, wherein the second aliphatic tail is saturated, and has from 6 carbon atoms to 12 carbon atoms in a linear arrangement.

10. The liposome of claim 9, wherein the second lipid is 1,2-diheptanoyl-sn-glycero-3-phosphocholine or 1,2-didecanoyl-sn-glycero-3-phosphocholine.

11. The liposome of claim 1, wherein the second aliphatic tail has from 4 carbon atoms to 15 carbon atoms less than the first aliphatic tail.

12. The liposome of claim 1, wherein the liposome does not comprise a polyethylene glycol group, a cholesterol group, or a polyethylene glycol group and cholesterol group.

13. The liposome of claim 1, wherein the mole percent ratio of the first lipid to the second lipid in the liposome is 70 or greater to 30 or less.

14. The liposome of claim 1, wherein the mole percent ratio of the first lipid to the second lipid is about 74 to about 25.

15. The liposome of claim 1, having a zeta potential of about −2 mV to about −9 mV.

16. The liposome of claim 15, having a zeta potential of about −3 to about −8 mV.

17. The liposome of claim 1, having a stretching modulus of less than about 250 mN/m by micropipette aspiration.

18. The liposome of claim 1, wherein molecular density of the bilayer gap is less than the molecular density at the bilayer surface.

19. A composition comprising a liposome of claim 1 and a cargo.

20. The composition of claim 19, wherein the cargo is a drug.

21. The composition of claim 20, wherein the drug is selected from an anti-cancer therapeutic, a vaccine, an anti-bacterial reagent, or an anti-fungal reagent.

22. The composition of claim 19, wherein the drug is an anti-cancer therapeutic.

23. A plurality of liposomes of claim 1, having a polydispersity index of about 0.01 to about 0.2.

24. The plurality of liposome of claim 23, having a polydispersity index of about 0.05 to about 0.1.

25. A method for delivering a drug to a tumor, the method comprising contacting a tumor with an effective amount of a composition of claim 20.

26. The method of claim 25, wherein the tumor is in a subject.

27. The method of claim 25, wherein the tumor is desmoplastic.

28. The method of claim 27, wherein the desmoplastic tumor is a tumor associated with breast cancer, colorectal cancer, prostate cancer, lung cancer, pancreatic cancer, renal cancer, ovarian cancer, or brain cancer.

29. A method for treating cancer in a subject in need thereof, comprising administering to the subject a therapeutically effective amount of a composition of claim 22.
